(19) United States
(12) Patent Application Publication
(10) Pub. No.: US 2026/0159850 A1
(43) Pub. Date: Jun. 11, 2026

(54) EPISTATIC TOMATO QTLS FOR INCREASING YIELD

(71) Applicant: YISSUM RESEARCH DEVELOPMENT COMPANY OF THE HEBREW UNIVERSITY OF JERUSALEM LT, Jerusalem (IL)

(72) Inventors: Shai Torgeman, Mazkeret Batya (IL); Havatzelet Pleban, Kiryat Ono (IL); Daniel Zamir, Gedera (IL)

(21) Appl. No.: 19/125,664

(22) PCT Filed: Oct. 30, 2023
(86) PCT No.: PCT/IL2023/051116

§ 371 Date: Apr. 29, 2025

Related U.S. Application Data
(60) Provisional application No. 63/420,591, filed on Oct. 30, 2022.

(51) Int. Cl. C12N 15/82

(52) U.S. Cl. CPC C12N 15/8261

(57) ABSTRACT

The present invention is in the field of tomato plant growth and development, particularly relating to positive non-additive (epistatic) interactions between quantitative trait loci (QTLs) conferring enhanced productivity and drought tolerance via heterosis in tomato plants, particularly of the species Solanum lycopersicum. According to certain aspects, the present invention provides a tomato crop plant comprising within its genome one or more exogenous genetic elements comprising a quantitative trait locus (QTL) derived from chromosome 1 (Chr-1 QTL) and a QTL derived from chromosome 7 (Chr-7 QTL) of Solanum pennellii Lost Accession LA5240.

The project leading to this application has received funding from the European Union's Horizon 2020 Research and Innovation Programme under grant agreements Nos. 677379, 727929 and 101000716 and from the Israel Science Foundation (ISF) no. 2365/20.

### FIELD OF THE INVENTION

The present invention is in the field of agriculture,, particularly relating to positive non-additive (epistatic) interactions between quantitative trait loci (QTLs) conferring enhanced productivity and drought tolerance via heterosis in plants, particularly tomato plants.

### BACKGROUND OF THE INVENTION

Heterosis, or plant hybrid vigor, is a phenomenon where hybrid plants display superior phenotypes compared to either of its inbred parent lines. Hybrid vigor was discovered in maize breeding nearly a century ago, and has subsequently been found to occur in many crop species (Duvick D N. 2001. Nat Rev Gent 2, 69-74). A large portion of the dramatic increase in agricultural output during the last half of the twentieth century has been attributed to the development and use of hybrid seed varieties in core crops.

The genetic basis of heterosis includes diverse mechanisms such as dominance, overdominance, epistasis and perhaps additional contributors (Birchler, J A et al., 2006. Proceedings of the National Academy of Sciences. 103, 12957-12958; Lippman, Z et al., 2007. Trends in genetics. 23, 60-66; Schnable, P S and Springer N M. 2013. Annual review of plant biology. 64, 71-88; Li, Zhi-Kang, et al. 2001. Genetics. 158, 1737-1753; Li, Lanzhi, et al. 2008. Genetics. 180, 1725-1742).

In mapping quantitative trait loci (QTL), mean values for a trait are compared in different genotypic groups in segregating populations or in genome wide association studies (GWAS) by ‘one marker at a time’, to generate plots of the effects on a trait and its significance along linkage groups. This approach made marker-assisted-selection a prerequisite for the success of plant breeding programs for desired traits, for example resistances to diseases and improved crop quality. Currently, thousands of SNPs can be scored, but typically the SNPs are analyzed mostly by ‘one at a time’ analysis along the genome. A deeper view in the analysis of complex traits would be to conduct genome-wide scans for QTLs that have small or no effect on a trait but a surprising outcome when combined with other QTL positions. According to Weinreich et al., epistasis can be regarded as a surprising phenotype when QTLs are combined, given the constituent QTL′ individual effects (Weinreich, D M et al., 2013. Current opinion in genetics & development. 23, 700-707).

Crop plants are often used to study complex traits since large populations of sessile individuals can be assayed in relatively uniform agriculture environments. Tomato (Solanum lycopersicum; 2n=2x=24) is the most abundant vegetable crop and a favorite plant for studying the genetic basis of yield associated traits, including epistasis (Soyk, S et al., 2020. Annual Review of Genetics. 54, 287-307). Tomato is also a leading crop in the deployment of wild species variation for the breeding of cutting-edge commercial hybrids (Zamir, D. 2001. Nature reviews genetics. 2, 983-989). One of the populations used for tomato genetics and breeding has been the S. pennellii (LA716) introgression lines (ILs), which provide a complete coverage of the S. pennellii. genome in a set of 76 lines, each containing a single genomic segment from the wild species (Eshed, Y and Zamir, D. 1995. Genetics. 141, 1147-1162). The ILs are nearly isogenic to an inbred processing tomato variety, and this greatly reduced the variation associated with QTL/QTL interactions compared to populations that segregate for the entire genome. Epistasis of yield-associated traits in S. pennellii ILs was searched by crossing ten different homozygous ILs and evaluating the 45 resulting hybrids compared to controls in the field (Eshed, Y, and Zamir, D. 1966. Genetics. 143, 1807-1817). For fruit weight and fruit sugar content (% Brix) traits, epistasis was found to be predominately of the less-than-additive mode, i.e., the phenotypic value of the double heterozygotes was lower than the sum of the effects of the single heterozygotes. For total fruit yield, however, epistasis followed the mode of more-than-additive, suggesting that it is possible to identify epistatic QTL that improve productivity beyond the sum of the effects of the individual QTL.

There are two main factors that limit the ability to detect significant epistasis in segregating populations: (1) typically, such populations include 100-200 individuals, which is a too low number to identify significant QTL-QTL interactions since the number of individuals that carry specific combinations of two (or more) genomic regions is very low; and (2) the ‘multiple testing penalty’ of all pairs of genomic regions (in the case of digenic interactions), which causes a strong downward adjustment of the statistical significance threshold (Mackay, Trudy F C. 2014. Nature Reviews Genetics. 15, 22-33). To overcome these limitations, a very large backcross inbred line (BIL) population, where each BC2S6 line can be traced to a different BC1 plant was developed. In this population, the wild donor for the BILs was an unexplored accession of S. pennellii that was rediscovered in the Gatersleben collection (Lost Accession, LA5240; Schmidt, Maximilian H-W., et al. 2017. The Plant Cell. 29, 2336-2348). This accession is completely self-compatible (a rare trait in this species) and importantly, does not carry the necrotic dwarf trait that is characteristic of S. pennellii LA716 and often interferes with the phenotypic evaluations (Eshed and Zamir, 1995, ibid). The Lost Accession was introgressed into a parental line suited for the processing tomato industry (determinate inbred, LEA).

Publication of the inventors of the present invention, published after the priority date of the present invention, describes the identification and validation of two epistatic QTLs that by themselves have no effect on yield but whose hybrids showed 20-50% heterosis in a consistent manner over a period of 4 years (Torgeman S and Zamir D. 2023. PNAS 120 (14): e2205787119; doi.org/10.1073/pnas.2205787119

There is a constant need for and it would be highly advantageous to have genetic elements that, when expressed in plants, particularly Solanaceae crop plant in general and tomato plants in particular, contribute to trait heterosis, particularly to an increase in plant crop productivity.

### SUMMARY OF THE INVENTION

The present invention discloses a combination of QTLs conferring to a hybrid tomato plant (Solanum lycopersicum) comprising same an improved crop productivity via epistatic interactions.

The present invention is based in part on the unexpected finding that tomato plants comprising a combination of two QTLs showed a yield increase of about 20 to 50% compared to plants comprising each of the QTL alone. The epistatic contribution of the QTL combination was observed in different genetic backgrounds and for a period of three years.

According to certain aspects, the present invention provides a tomato crop plant comprising within its genome one or more exogenous genetic elements comprising a quantitative trait locus (QTL) derived from chromosome 1 (Chr-1 QTL) and a QTL derived from chromosome 7 (Chr-7 QTL) of Solanum pennellii Lost Accession LA5240, wherein each of the Chr-1 QTL and the Chr-7 QTL is present in the tomato plant in a heterozygous form and wherein the combination of QTLs confers an increase in the tomato crop plant productivity compared to the productivity of a corresponding tomato crop plant comprising none or one of said QTLs.

According to certain embodiments, both of the Chr-1 QTL and the Chr-7 QTL are present on a single genetic element.

According to certain embodiments, each of the Chr-1 QTL and the Chr-7 QTL is present on a separate genetic element. According to these embodiments, the genetic element may comprise or consists of the QTL.

According to certain embodiments, the plant is genetically engineered to comprise the at least one exogenous genetic element.

According to certain embodiments, the Chr-1 QTL comprises a polynucleotide comprising a nucleic acid sequence having at least 96% identity to the nucleic acid sequence of S. pennellii LA5240 chromosome 1 from about 100.789953 cM to about 127.0420104 cM or a part thereof.

According to certain embodiments, the Chr-1 QTL comprises a polynucleotide comprising a nucleic acid sequence having at least 96% identity to the nucleic acid sequence of S. pennellii LA5240 chromosome 1 from position 92541344 bp (1:9254134) to position 98424021 bp (1:98424021) or a part thereof.

According to certain embodiments, the polynucleotide sequence of S. pennellii LA5240 chromosome 1 from position 92555142 bp to position 92556268 bp is at least 96% identical to the nucleic acid sequence set forth in SEQ ID NO:1. According to certain embodiment, the polynucleotide sequence of S. pennellii LA5240 chromosome 1 from position 92555142 bp to position 92556268 comprises the nucleic acid sequence set forth in SEQ ID NO:1.

According to certain embodiments, the polynucleotide sequence of S. pennellii LA5240 chromosome 1 from position 98423130 bp to position 98424009 is at least 96% identical to the nucleic acid sequence set forth in SEQ ID NO:2. According to certain embodiments, the polynucleotide sequence of S. pennellii LA5240 chromosome 1 from position 98423130 bp to position 98424009 comprises the nucleic acid sequence set forth in SEQ ID NO:2.

According to certain additional or alternative embodiments, the Chr-1 QTL comprises a nucleic acid marker (designated herein SSL2.50CH01_95261222), wherein the marker is amplified by a pair of primers comprising SEQ ID NO:3 and SEQ ID NO:4 and wherein said marker is not amenable to digestion by EcoRI.

According to certain embodiments, the marker comprises the nucleic acid sequence set forth in SEQ ID NO:5, having a length is of 729 nucleotides.

According to certain embodiments, the Chr-7 QTL comprises a polynucleotide comprising a nucleic acid sequence having at least 96% identity to the nucleic acid sequence of S. pennellii LA5240 chromosome 7 from about 53.3003069 cM to about 56.96038414 cM or a part thereof.

According to certain embodiments, the Chr-7 QTL comprises a polynucleotide comprising a nucleic acid sequence having at least 96% identity to the nucleic acid sequence of S. pennellii LA5240 chromosome 7 from position 66067020 bp (7:66067020) to position 66509915 bp (7:66509915) or to a part thereof.

According to certain embodiments, the polynucleotide sequence of S. pennellii LA5240 chromosome 7 from position 66072049 bp to 66/073,605 bp is at least 96% identical to the nucleic acid sequence set forth in SEQ ID NO:6. According to certain embodiments, the polynucleotide sequence of S. pennellii LA5240 chromosome 7 from position 66072049 bp to 66/073,605 bp comprises the nucleic acid sequence set forth in SEQ ID NO:6.

According to certain embodiments, the polynucleotide sequence of S. pennellii LA5240 chromosome 7 from position 66505354 bp to position 66509027 bp is at least 96% identical to the nucleic acid sequence set forth in SEQ ID NO:7. According to certain embodiments, the polynucleotide sequence of S. pennellii LA5240 chromosome 7 from position 66505354 bp to position 66509027 bp comprises the nucleic acid sequence set forth in SEQ ID NO:7.

According to certain embodiments, the Chr-7 QTL comprises a nucleic acid marker (designated herein SSL2.50CH07_65737800) wherein the marker is amplified by a pair of primers comprising SEQ ID NO:8 and SEQ ID NO:9 and wherein said marker is not amenable to digestion by HphI.

According to certain embodiments, the marker comprises the nucleic acid sequence set forth in SEQ ID NO:10, having a length is of 528 nucleotides.

According to certain embodiments, the Chr-1 QTL is incorporated within chromosome 1 of the recipient tomato plant.

According to certain embodiments, the Chr-7 QTL is incorporated within chromosome 7 of the recipient tomato plant.

According to certain embodiments, the tomato plant is of the Solanum lycopersicum species.

According to certain exemplary embodiments, the Chr-1 QTL is incorporated at a position of from about 100.789953 cM to about 127.0420104 cM on the S. lycopersicum chromosome 1, and the Chr-7 QTL is incorporated at a position of from about 53.3003069 cM to about 56.96038414 cM on said S. lycopersicum chromosome 7.

According to certain embodiments, the increase in crop productivity comprises an increase in at least one of yield associated traits. According to certain exemplary embodiments, the yield associated trait is selected from the group consisting of inflorescence number, fruit number, plant weight, drought tolerance, and any combination thereof. Each possibility represents a separate embodiment of the present invention. According to certain exemplary embodiments, the increase in inflorescence number and/or fruit number, is per plant.

According to certain embodiments, the tomato plant comprising the at least one genetic element comprising the QTL combination is devoid of deleterious genetic drags originated from the S. pennellii chromosome.

According to certain embodiments, the tomato plant comprising the combination of Chr-1 QTL and Chr-7 QTL is equivalent to a corresponding tomato plant lacking the introduced QTLs in at least one of fruit taste, resistance to abiotic stresses, resistance to pathogens, and any combination thereof.

According to certain exemplary embodiments, the QTLs are introduced into a S. lycopersicum elite crop plant. It is to be understood that the tomato plant of the S. lycopersicum species of the present invention is a crop plant, but is not restricted to a specific line and/or variety.

Seeds, cuttings, and any other plant parts that can be used for propagation, including isolated cells and tissue cultures are also encompassed within the scope of the present invention. It is to be understood that the plant produced from said seeds or other propagating material comprises the QTL combination conferring the enhanced crop productivity.

The present invention discloses hitherto unknown epistatic associations between a QTL present on chromosome 1 and a QTL present on chromosome 7 within S. pennellii genome. Presence of each of the QTL alone in an S. lycopersicum species has no effect on crop productivity, particularly on fruit yield and plant weight, while the combination of both significantly enhanced the S. lycopersicum crop productivity.

According to certain aspects, the present invention provides an isolated polynucleotide comprising a nucleic acid sequence having at least 96% identity to the nucleic acid sequence of S. pennellii chromosome 1 between positions 100.789953 cM and 127.0420104 cM or a part thereof.

According to certain embodiments, the isolated polynucleotide comprises a nucleic acid sequence derived from a segment of chromosome 1 of S. pennellii Lost Accession LA5240 or a part thereof. According to certain embodiments, the segment is starting at position 100.789953 cM and ending at position 127.0420104 cM of said chromosome 1. According to certain embodiments, the segment is starting at position 92541344 bp and ending at position 98424021 bp of said chromosome 1.

According to certain embodiments, the polynucleotide comprises a nucleic acid sequence having at least 96% identity to a nucleic acid sequence selected from the group consisting of SEQ ID NO:1, SEQ ID NO:2 and a combination thereof. According to certain embodiments, the polynucleotide comprises the nucleic acid sequence set forth in the nucleic acid sequence selected from the group consisting of SEQ ID NO:1, SEQ ID NO: 2 and a combination thereof.

According to certain exemplary embodiments, the isolated polynucleotide comprises at least one marker amplified by a pair of primers comprising the nucleic acid sequence set forth in SEQ ID NO:3 and SEQ ID NO:4, wherein the marker is not amenable to digestion by EcoRI. According to certain embodiments, the marker comprises the nucleic acid sequence set forth in SEQ ID NO:5

According to certain additional aspects, the present invention provides an isolated polynucleotide comprising a nucleic acid sequence having at least 96% identity to the nucleic acid sequence of S. pennellii chromosome 7 between positions 53.3003069 cM and 56.96038414 cM.

According to certain embodiments, the isolated polynucleotide comprises a nucleic acid sequence derived from a segment of chromosome 7 of S. pennellii Lost Accession LA5240 or a part thereof. According to certain embodiments, the segment is starting at position 53.3003069 cM and ending at position 56.96038414 cM of said chromosome 7. According to certain embodiments, the segment is starting at position 66067020 bp and ending at position 66509915 bp of said chromosome 7.

According to certain embodiments, the polynucleotide comprises a nucleic acid sequence having at least 96% identity to a nucleic acid sequence selected from the group consisting of SEQ ID NO:6, SEQ ID NO:7 and a combination thereof. According to certain embodiments, the polynucleotide comprises the nucleic acid sequence selected from the group consisting of SEQ ID NO:6, SEQ ID NO:7 and a combination thereof.

According to certain exemplary embodiments, the isolated polynucleotide comprises at least one marker amplified by a pair of primers comprising the nucleic acid sequence set forth in SEQ ID NO:8 and SEQ ID NO:9, wherein the marker is not amenable to digestion by HphI. According to certain embodiments, the marker comprises the nucleic acid sequence set forth in SEQ ID NO:10.

DNA constructs and expression vectors comprising the isolated polynucleotide are also encompassed within the scope of the present invention. According to certain embodiments, the DNA construct further comprises at least one regulatory element operably linked to the isolated polynucleotides.

According to yet further certain aspects, the present invention provides a method for producing a tomato crop plant having an enhanced crop productivity, the method comprising introducing into the tomato plant genome one or more genetic elements comprising Chr-1 QTL and Chr-7 QTL, wherein a heterozygous expression of the combination of the QTLs confers an increase in crop productivity compared to a corresponding tomato plant comprising none or one of said QTLs.

According to certain embodiments, the Chr-1 QTL and the Chr-7 QTL are present on a single genetic element.

According to certain exemplary embodiments, each of the Chr-1 QTL and the Chr-7 QTL is present on a separate genetic element.

According to certain embodiments, the Chr-1 QTL or the genetic element comprising same is introduced into one allele of chromosome 1 of the tomato plant and the Chr-7 QTL or the genetic element comprising same is introduced into one allele of chromosome 7 of the tomato plant. The tomato plant is of the Solanum lycopersicum species.

According to certain aspects, the present invention provides a method for identifying and selecting a tomato plant characterized by an increased crop productivity, comprising the steps of:
a. providing a plurality of tomato crop plants;
b. examining a nucleic acid sample obtained from each of the plurality of the tomato crop plant for the presence of a Chr-1 QTL and Chr-7 QTL, thereby identifying a population comprising both QTLs; and
c. examining the population comprising both QTLs for enhanced crop productivity compared to a control plant or to a pre-determined productivity score value;
thereby selecting a tomato plant characterized by an increased crop productivity compared to the control plant.

According to certain embodiments, the control plant is a tomato plant comprising none or one of the QTLs.

The QTLs and genetic elements are as described hereinabove.

Any method as is known to a person skilled in the art can be used to introduce the combination of Chr-1 QTL and Chr-7 QTL to a tomato plant, particularly a tomato plant of the S. lycopersicum species.

According to certain embodiments, each of the genetic elements is introduced by introgression from a donor tomato plant to different recipient tomato plant, thereby producing a tomato plant comprising Chr-1 QTL and a tomato plant comprising Chr-7 QTL. According to these embodiments, the method further comprises crossing the recipient plants to form the tomato plant comprising the epistatic heterozygous combination of QTLs.

According to certain embodiments, the at least one genetic element is an isolated nucleic acid introduced into a recipient tomato plant by a method known to a person skilled in the art. According to certain embodiments, the isolated nucleic acid is introduced by transformation. According to certain additional or alternative embodiments, the at least one isolated nucleic acid is introduced by gene-editing. According to some embodiments, the transformed tomato plant comprising each or the combination of Chr-1 QTL and Chr-7 QTL is self- or cross-pollinated to produce a plant comprising the combination of Chr-1 QTL and Chr-7 QTL as described hereinbelow.

According to certain embodiments, the isolated nucleic a acid comprising each of the QTLs or a combination thereof is introduced into one allele in the genome of the recipient tomato crop plant.

According to certain embodiments, selecting a tomato plant comprising the combination of QTLs is performed by detecting the presence of the QTLs within the genome of the tomato (S. lycopersicum) plant. Any method as is known in the art can be used to detect the QTL or a part thereof.

According to certain exemplary embodiments, detection is performed by identifying the markers located within the QTLs as described herein.

According to additional or alternative exemplary embodiments, detection is performed by phenotypically identifying tomato plants having an increase in crop productivity compared to the parent tomato plant, to a control plant or to a pre-determined productivity value.

According to certain exemplary embodiments, the tomato crop plant is of the species Solanum lycopersicum.

It is to be understood that any combination of each of the aspects and the embodiments disclosed herein is explicitly encompassed within the disclosure of the present invention.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

FIG. 1A to FIG. 1D demonstrate the genomic composition of the biparental BILs. FIG. 1A: Plots of the physical (Mbp) and genetic (cM) distances of the 7699 SPET markers of the 12 tomato chromosomes. The arrow points to the largest gap detected in chromosome 3 (11 Mbp). FIG. 1B: Frequency distribution of the number of wild species introgressions per BIL. FIG. 1C: The prevalence of the S. pennellii alleles in BC2S6 relative to the expected value of 12.5%. FIG. 1D: A picture of the experimental plots in Akko showing the replicated single young plants each covering an area of 1 m².

FIG. 2A to FIG. 2D show the identification of single yield QTL in the BILs and BILHs. FIG. 2A: Frequency distribution of total yield (TY, kg) in the 1389 homozygote BILs. FIG. 2B: Frequency distribution of TY (kg) in the 1233 heterozygous BILH. FIG. 2C: Single marker analysis of TY (kg) in the BILs relative to the TY of LEA. LOD scores for each of the marker effects was calculated by Haley-Knott regression and the LOD threshold was determined by 1000 permutation tests. FIG. 2D: Single marker analysis of TY (kg) in the BILHs relative to the TY of LEA. LOD scores for each of the marker effects was calculated by Haley-Knott regression and the LOD threshold was determined by 1000 permutation tests.

FIG. 3A to FIG. 3F demonstrate the distribution of the yield-associated traits in the BIL and BILHs. FIGS. 3A and 3D: Frequency distribution for plant weight (PW) in the BILs and BILHs, respectively. FIGS. 3B and 3E: Frequency distribution for fruit weight (FW) in the BILs and BILHs, respectively. FIG. 3C and FIG. 3F: Frequency distribution for Brix (BX %) in the BILs and BILHs, respectively.

FIG. 4A to FIG. 4F show single marker analysis of yield-associated traits in the BIL and BILHs. FIGS. 4A and 4B: QTL analysis for plant weight (PW) in the BILs and the BILHs. FIG. 4C and FIG. 4D: QTL analysis of fruit weight (FW) in the BILs and BILHs. FIGS. 4E and 4F: QTL analysis for total soluble solids (BX %) in the BIL and BILH. LOD scores for each of the marker effects was calculated by Haley-Knott regression and the LOD threshold was determine by 1000 permutation tests. All the effect values are in percent of the value obtained for the control tomato “LEA” plants.

FIG. 5A to FIG. 5C show validation analysis of chromosome 4 for the effect on total yield. FIG. 5A: Initial detection of the yield QTL that increases TY by 20% in BILH (Akko, Israel 2018). FIG. 5B: The BILH heterozygous for the chromosome 4 QTL was crossed to eight processing tomato inbred lines and the progenies were genotyped for the chromosome 4 marker. The two genotypic groups were compared with a t-test and * indicates a significant difference (P<0.05). FIG. 5C: A test of the pooled total yield (TY) data from all the eight genetic backgrounds shows an 18% yield increase due to the chromosome 4 QTL.

FIG. 6A to FIG. 6D show the identification and validation of epistatic QTL. FIG. 6A: Two-dimensional QTL scan of the BILs and BILHs for plant weight (PW), fruit weight (FW), Brix (BX) and total yield (TY) in dry conditions (Akko, Israel, 2018). LOD fv1>3¹⁶ was used to indicate significant epistasis. FIG. 6B: Whole genome circus epistasis plot. White segments on each of the chromosomes signify the euchromatic regions and gray segments signify the heterochromatin. FIG. 6C: Yield epistasis (Akko, Israel, 2018) detected for markers on chromosomes 1 (SSL2.50CH01_95261222) and 7 (SSL2.50CH07_65737800) showing the number (N) of plants in each of the genotypic groups: 1) Homozygous for the cultivated tomato alleles in chromosomes 1 and 7 (1_1). 2) Heterozygous for the chromosome 1 introgression (2_1). 3) Heterozygous for the chromosome 7 introgression (1_2). 4) Heterozygous for both introgressions (2_2). Genotypic groups' means showing the same letters are not significantly different at the 5% level based on the Tukey-Kramer test. FIG. 6D: A validation test of chromosomes 1 and 7 epistasis in F2 progenies of the double heterozygous BILH (Akko, Israel, 2020). Markers, genotypic groups, and statistical tests are the same as in FIG. 6C.

FIG. 7A to FIG. 7E demonstrate epistatic QTL for yield heterosis. FIG. 7A: A LEA background hybrid, heterozygous for the QTL on chromosomes 1 and 7 was crossed to nine different processing tomato inbred lines to produce progenies of four genotypic groups which were assayed for total yield in Akko, Israel, 2021. Total yield (TY) of the double heterozygous hybrids (2_2) with inbred lines10640 and 10643 was significantly higher than the other genotypes (5% level based on Tukey-Kramer test). FIG. 7B: A test of the pooled total yield (TY) from all the nine genetic backgrounds (FIG. 7A) shows that the double introgression hybrids had the highest yield of the four genotypes. Means with the same letters are not significantly different at the 5% level based on the Tukey-Kramer test. FIG. 7C: A test of the pooled plant weight (PW) data from all the nine genetic backgrounds shows that the double introgression hybrids had the largest PW. Means with the same letters are not significantly different at the 5% level based on the Tukey-Kramer test. FIGS. 7D and 7E: To test whether the epistatic QTL on chromosome 1 and 7 drives yield heterosis, two homozygous BILs, each carrying a single QTL, were selected and crossed to LEA and to each other. The yield of the two BILs, their hybrids with LEA and the double introgression hybrid was tested in Akko 2020 in well irrigated conditions (FIG. 7D) and under drought conditions (FIG. 7E). Means with the same letters are not significantly different at the 5% level based on the Tukey-Kramer test.

FIG. 8A to FIG. 8D demonstrate the validation of the epistatic QTL in plots of commercial plant density. Yield components (Akko 2022) measured in 5 m² plots with 12 plants each. The plots of the four genotypic groups are presented with respect to the markers on chromosomes 1 (SSL2.50CH01_95261222) and 7 (SSL2.50CH07_65737800): 1) Homozygous for the cultivated tomato alleles in chromosomes 1 and 7 (1_1). 2) Heterozygous for the chromosome 1 introgression (2_1). 3) Heterozygous for the chromosome 7 introgression (1_2). 4) Heterozygous for both introgressions (2_2). Genotypic groups' means showing the same letters are not significantly different at the 5% level based on the Tukey-Kramer test. The traits measured were plant weight (FIG. 8A), total yield (FIG. 8B), fruit weight (FIG. 8C) and the estimated fruit number (FIG. 8D).

FIG. 9A to FIG. 9D demonstrate the validation of the epistatic QTL in wide spacing. Yield components (Akko 2022) measured using replications of 1 plant per m². The results of the four genotypic groups are presented with respect to the markers on chromosomes 1 (SSL2.50CH01_95261222) and 7 (SSL2.50CH07_65737800): 1) Homozygous for the cultivated tomato alleles in chromosomes 1 and 7 (1_1). 2) Heterozygous for the chromosome 1 introgression (2_1). 3) Heterozygous for the chromosome 7 introgression (1_2). 4) Heterozygous for both introgressions (2_2). Genotypic groups' means showing the same letters are not significantly different at the 5% level based on the Tukey-Kramer test. The traits measured were plant weight (FIG. 9A), total yield (FIG. 9B), fruit weight (FIG. 9C) and the estimated fruit number (FIG. 9D).

FIG. 10A to FIG. 10B show number of inflorescences. FIG. 10A: Number of inflorescences along the main stem of plants heterozygous for the heterotic QTL on chromosomes 1 and 7 in the LEA background (2_2) compared to the genotypes 1_1; 1_2 and 2_1. FIG. 10B: Number of inflorescences along the main stem of the plants heterozygous for the heterotic QTL on chromosomes 1 and 7 in the 10640 and 10643 background compared to the genotypes 1_1; 1_2 and 2_1.

FIG. 11A to FIG. 11B is a schematic presentation of fine mapping scheme for the QTL involved in the epistasis. For fine mapping of the epistatic QTL on chromosome 1 and 7, the two introgressions that create the epistasis and the mapped recombinant BILs for each of the chromosomes are used. The BILH for the chromosome 1 QTL (FIG. 11A) are crossed to homozygous BILs that are recombinant in chromosome 7. The progeny of such crosses produces nearly isogenic hybrids of two genotypes: with or without the QTL on chromosome 1. The yield of the isogenic hybrids is compared and if the hybrids with the chromosome 1 QTL have higher yield, the recombined segment of the BIL on chromosome 7 carries the second generates heterosis. A comparison of the values of the two genotypic groups using multiple recombinants BILs indicate the location of the chromosome 7 QTL. To map the QTL on chromosome 1 which is involved in the interaction, the BILH of the chromosome 7 QTL (FIG. 11B) is crossed to recombinants BILs of chromosome 1 and the scheme described above is followed.

FIG. 12 shows companion leaves at the base of a leaf of S. pennellii.

### DETAILED DESCRIPTION OF THE INVENTION

### Definitions

The terms “comprise”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a part” with reference to a polynucleotide may include a plurality of polynucleotide parts, including mixtures thereof.

The term “about” as used herein refers to a numeric value ±10%.

As used herein, unless specifically indicated otherwise, the word “or” is used in the inclusive sense of “and/or” and not the exclusive sense of “either/or.”

The term “plant” is used herein in its broadest sense. It also refers to a plurality of plant cells that are largely differentiated into a structure that is present at any stage of a plant's development. Such structures include, but are not limited to, a root, stem, shoot, leaf, flower, petal, fruit, etc. According to certain exemplary embodiments, the plant is a tomato plant. According to certain exemplary embodiments, the tomato plant is Solanum lycopersicum crop plant.

As used herein, the term “crop plant” denote a plant having a biological status other than a “wild” status, which “wild” status indicates the original non-cultivated or natural state of a plant or accession. The term “crop plant” (for cultivated plants) includes, but is not limited to, semi-natural, semi-wild, traditional cultivar, landrace, breeding material, research material, breeder's line, synthetic population, hybrid, founder stock/base population, inbred line (parent of hybrid cultivar), segregating population, mutant/genetic stock, and advanced/improved cultivar. The term as used herein includes registered as well as non-registered lines.

As used herein, the term “plant part” typically refers to a part of the tomato plant, including single cells and cell tissues such as plant cells that are intact in plants, cell clumps and tissue cultures from which tomato plants can be regenerated. Examples of plant parts include, but are not limited to, single cells and tissues from pollen, ovules, leaves, embryos, roots, root tips, anthers, flowers, fruits, stems, shoots, and seeds; as well as pollen, ovules, leaves, embryos, roots, root tips, anthers, flowers, fruits, stems, shoots, scions, rootstocks, seeds, protoplasts, calli, and the like.

The term “locus” (plural “loci”) is defined herein as the position that a given gene or genes occupies on a chromosome of a given species.

The term “quantitative trait locus” or “QTL” is used herein in its art-recognized meaning. In certain exemplary embodiments, the term “QTL” refers to a region located on a particular chromosome of Solanum that is associated with at least one gene or at least a regulatory region, i.e., a region of a chromosome that controls the expression of one or more genes. A QTL may for instance comprise one or more genes of which the products confer the increase in crop production. Alternatively, a QTL may for instance comprise regulatory genes or sequences of which the products influence the expression of genes on other loci in the genome of the plant thereby conferring an increase in crop productivity. The QTL of the present invention may be defined by indicating its genetic location in the genome of the respective S. pennellii accession using one or more molecular genomic markers. One or more markers, in turn, indicate a specific locus. Distances between loci are usually measured by frequency of crossing-over between loci on the same chromosome and expressed as centimorgan (cM). The further apart two loci are, the more likely that a crossover will occur between them. Conversely, if two loci are close together, a crossover is less likely to occur between them. As a rule, one centimorgan (Kosambi map function (cM)) is approximately equal to 1% recombination between loci (markers). When a QTL can be indicated by multiple markers the genetic distance between the end-point markers is indicative of the size of the QTL. According to certain currently exemplary embodiments, the term “Chr-1 QTL” refers to a QTL derived from chromosome 1 of S. pennellii Lost Accession LA5240; and the term “Chr-7 QTL” refers to a QTL derived from chromosome 7 of S. pennellii Lost Accession LA5240 as described herein.

When reference is made to particular sequence listings, such reference is to be understood to also encompass sequences that substantially correspond to its complementary sequence, as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

As used herein, “S. pennellii Lost Accession LA5240” refers to a wild, green-fruited tomato species also designated S. pennellii Accession LYC1722, genome of which has been characterized by Maximilian et al. (Maximilian H.-W. et al., 2017. The Plant Cell, 29(10), 2336-2348; doi.org/10.1105/tpc.17.00521).

The term “natural genetic background” is used herein to indicate the original genetic background of a QTL. According to certain embodiments of the present invention, such a background is the genome of Solanum pennellii Lost Accession LA5240. Accordingly, S. pennellii Lost Accession LA5240 represents the natural genetic background of the QTLs of the invention. A method that involves the transfer of a DNA comprising the QTLs or a part thereof, from chromosome 1 and chromosome 7 of S. pennellii to the same or different position on the corresponding chromosome of another plant, particularly S. lycopersicum tomato plant, will result in that QTLs or part thereof not being in its natural genetic background. Seeds of S. pennellii Lost Accession LA5240 are available at C. M. Rick Tomato Genetics Resource Center.

The term “epistasis” or “epistatic” as used herein refers to a non-linear (non-additive) interaction of genes.

The term “exogenous” as used herein, particularly regarding an exogenous genetic element, refers to a heterologous nucleic acid sequence which is not naturally expressed within the plant (e.g., a nucleic acid sequence from a different plant species or variety).

According to certain embodiments of the invention, the exogenous genetic element comprises a nucleic acid sequence derived from Solanum pennellii, and is expressed in a Solanum lycopersicum. According to certain aspects of the invention, the exogenous polynucleotide is introduced into the plant in a stable manner, so as to produce a ribonucleic acid (RNA) molecule and/or a polypeptide molecule. According to certain embodiments, the exogenous polynucleotide is introduced into the plant by genetic engineering tools.

The term “heterozygous” as is used herein means a genetic condition existing when different alleles reside at corresponding loci on homologous chromosomes.

The term “homozygous” as is used herein, means a genetic condition existing when identical alleles reside at corresponding loci on homologous chromosomes.

As used herein, the term “hybrid” refers to any offspring of a cross between two genetically unlike individuals, including but not limited to the cross between two inbred lines.

As used herein, the term “population” refers to a genetically heterogeneous collection of plants sharing a common genetic derivation.

The terms “genetic engineering”, “introduction”, “transformation” and “genetic modification” are all used herein for the transfer of isolated and cloned genetic elements, which can comprise non-coding and coding regions, into the DNA, usually the chromosomal DNA or genome, of another organism, or to the modification of a gene within the plant genome.

As used herein the term “polynucleotide” refers to a single or double stranded nucleic acid sequence which is isolated and provided in the form of an RNA sequence, a complementary polynucleotide sequence (cDNA), a DNA sequence and/or a composite polynucleotide sequence (e.g., a combination of the above).

The term “isolated” refers to at least partially separated from the natural environment e.g., from a plant cell.

The terms “molecular marker” or “DNA marker” or “marker” are used herein interchangeably and refer to a molecular indicator that is used in methods for visualizing differences in characteristics of nucleic acid sequences. Examples of such indicators are Genotype By Sequencing (GBS markers), diversity array technology (DArT) markers, restriction fragment length polymorphism (RFLP) markers, amplified fragment length polymorphism (AFLP) markers, single nucleotide polymorphisms (SNPs), insertion mutations, microsatellite markers, sequence-characterized amplified regions (SCARs), cleaved amplified polymorphic sequence (CAPS) markers or isozyme markers or combinations of the markers described herein which defines a specific genetic and chromosomal location. According to some embodiments, the DNA markers are based on the Single Primer Enrichment technology (SPET). Like DNA chips, SPET is a targeted genotyping technology, relying on the sequencing of a region flanking a primer.

According to certain aspects, the present invention provides a tomato plant of the Solanum lycopersicum species crop plant comprising within its genome at least one genetic element comprising a QTL derived from chromosome 1 (Chr-1 QTL) and a QTL derived from chromosome 7 (Chr-7 QTL) of Solanum pennellii Lost Accession LA5240, wherein each of the Chr-1 QTL and the Chr-7 QTL is present in the tomato plant in a heterozygous form and wherein the combination of QTLs confers an increase in crop productivity compared to a plant comprising none or one QTL.

The genetic unit “QTL” indicates a region on the genome that is directly related to a phenotypic quantifiable trait. The present invention discloses epistatic interaction between Chr-1 QTL and Chr-7 QTL leading to phenotypes of improved plant productivity, particularly an increase in yield associated traits including, but not limited to, inflorescence number, fruit number, fruit weight, and plant weight. QTL differs from the genetic unit “gene”, on which the phenotypic expression depends, comprising, in additional to genes, a number of factors.

According to certain embodiments, the Chr-1 QTL comprises a polynucleotide comprising a nucleic acid sequence having at least 96%, at least 97%, at least 97.5%, at least 98%, at least 98.5%, at least 99%, at least 99.5%, or more identity to the nucleic acid sequence of S. pennellii LA5240 chromosome 1 from about 100.789953 cM to about 127.0420104 cM or to a part thereof.

According to certain embodiments, the Chr-1 QTL comprises a polynucleotide comprising a nucleic acid sequence having at least 96%, at least 97%, at least 97.5%, at least 98%, at least 98.5%, at least 99%, at least 99.5%, or more identity to the nucleic acid sequence of S. pennellii LA5240 chromosome 1 from position 92541344 bp (1:9254134) to position 98424021 bp (1:98424021) or to a part thereof.

According to certain embodiments, the polynucleotide sequence of S. pennellii LA5240 chromosome 1 from position 92555142 bp to position 92556268 bp is at least 96%, at least 97%, at least 97.5%, at least 98%, at least 98.5%, at least 99%, at least 99.5%, or more identical to the nucleic acid sequence set forth in SEQ ID NO:1. According to certain embodiment, the polynucleotide sequence of S. pennellii LA5240 chromosome 1 from position 92555142 bp to position 92556268 comprises the nucleic acid sequence set forth in SEQ ID NO:1.

According to certain embodiments, the polynucleotide sequence of S. pennellii LA5240 chromosome 1 from position 98423130 bp to position 98424009 is at least 96%, at least 97%, at least 97.5%, at least 98%, at least 98.5%, at least 99%, at least 99.5%, or more identical to the nucleic acid sequence set forth in SEQ ID NO:2. According to certain embodiments, the polynucleotide sequence of S. pennellii LA5240 chromosome 1 from position 98423130 bp to position 98424009 comprises the nucleic acid sequence set forth in SEQ ID NO:2.

As used herein, “sequence identity” or “identity” in the context of two nucleic acid sequences includes reference to the residues in the two sequences which are the same when aligned.

Identity (e.g., percent homology) can be determined using any homology comparison software, including for example, the BlastN, BlastX or Blastp software of the National Center of Biotechnology Information (NCBI) such as by using default parameters. A widely used and accepted computer program for performing sequence alignments is CLUSTALW v1.6 (Thompson, et al. Nucl. Acids Res., 22:4673-4680, 1994).

According to some embodiments of the invention, the identity is a global identity, i.e., over the entire nucleic acid sequences of the invention and not over portions thereof. According to some embodiments of the invention, the identity is a partial identity, i.e., over fragment or fragments of the nucleic acid sequences of the invention and not over the entire sequence, as described herein.

According to certain additional or alternative embodiments, the Chr-1 QTL comprises a nucleic acid marker (designated herein SSL2.50CH01_95261222) wherein the marker is amplified by a pair of primers comprising SEQ ID NO:3 and SEQ ID NO:4 and wherein said marker is not amenable to digestion by EcoRI.

According to certain embodiments, the marker comprises the nucleic acid sequence set forth in SEQ ID NO:5, having a length is of 729 nucleotides.

According to certain embodiments, the marker corresponds to the nucleic acid sequence of S. pennellii LA5240 chromosome 1 from position 94486205 bp to position 94486933 bp.

According to certain embodiments, the Chr-7 QTL comprises a polynucleotide comprising a nucleic acid sequence having at least 96%, at least 97%, at least 97.5%, at least 98%, at least 98.5%, at least 99%, at least 99.5%, or more identity to the nucleic acid sequence of S. pennellii LA5240 chromosome 7 from about 53.3003069 cM to about 56.96038414 cM or to a part thereof.

According to certain embodiments, the Chr-7 QTL comprises a polynucleotide comprising a nucleic acid sequence having at least 96%, at least 97%, at least 97.5%, at least 98%, at least 98.5%, at least 99%, at least 99.5%, or more identity to the nucleic acid sequence of S. pennellii LA5240 chromosome 7 from position 66067020 bp (7:66067020) to position 66509915 bp (7:66509915) or to a part thereof.

According to certain embodiments, the polynucleotide sequence of S. pennellii LA5240 chromosome 7 from position 66072049 bp to 66/073,605 bp is at least 96%, at least 97%, at least 97.5%, at least 98%, at least 98.5%, at least 99%, at least 99.5%, or more identical to the nucleic acid sequence set forth in SEQ ID NO:6. According to certain embodiments, the polynucleotide sequence of S. pennellii LA5240 chromosome 7 from position 66072049 bp to 66/073,605 bp comprises the nucleic acid sequence set forth in SEQ ID NO:6.

According to certain embodiments, the polynucleotide sequence of S. pennellii LA5240 chromosome 7 from position 66505354 bp to position 66509027 bp is at least 96%, at least 97%, at least 97.5%, at least 98%, at least 98.5%, at least 99%, at least 99.5%, or more identical to the nucleic acid sequence set forth in SEQ ID NO:7. According to certain embodiments, the polynucleotide sequence of S. pennellii LA5240 chromosome 7 from position 66505354 bp to position 66509027 bp comprises the nucleic acid sequence set forth in SEQ ID NO:7.

According to certain embodiments, the Chr-7 QTL comprises a nucleic acid marker (designated herein SSL2.50CH07_65737800) wherein the marker is amplified by a pair of primers comprising SEQ ID NO:8 and SEQ ID NO:9 and wherein said marker is not amenable to digestion by HphI.

According to certain embodiments, the marker comprises the nucleic acid sequence set forth in SEQ ID NO:10, having a length is of 528 nucleotides.

According to certain embodiments, the marker corresponds to the nucleic acid sequence of S. pennellii LA5240 chromosome 7 from position 66356170 bp to 66/356,697 bp.

As used herein, the term “increase in crop productivity” refers top increase in at least one of yield associated trait, including, but not limited to, inflorescence number, fruit number, plant weight, drought tolerance, and any combination thereof. The increase in the crop productivity is measured compared to the crop productivity of a corresponding plant comprising only single QTL or none of the QTL, or to a pre-determined crop productivity value. The pre-determined productivity value may be based on an average crop production of a corresponding tomato cultivar. As used herein, “an increase” refers to at least 10%, 15%, 20%, 25%, 30%, 35%, 40%, 45%, 50%, 55%, 60%, 65%, 70%, 75%, 80%, 85%, 90%, 95%, 100%, or more in at least one yield associated trait. According to certain embodiments, the increase in crop productivity is at least in the range of from about 10% to about 60%. According to certain embodiments, the increase in crop productivity is at least in the range of from about 20% to about 50%. According to certain embodiments, the increase is in the fruit number, resulting in an increase in the fruit yield.

Recent tomato studies demonstrated the existence of epistasis in tomato. Soyk et al. (Soyk S. et al., 2017. Cell. 169, 1142-1155) investigated epistasis between two independently segregating homologous MADS box mutations in tomato. One of the mutations promotes enlargement of a leaf-like organ on fruit sepals and was favored during domestication due to its effect of increasing fruit size. The second recessive mutation eliminates the flower abscission zone leading to “jointless” fruit stems that improve the efficiency of the mechanical harvest. Lines that combine both mutations in a homozygous state produce an extremely over-branched inflorescence with partial sterility and thus the yield is low. However, when the highly branched inflorescence lines were crossed with a wildtype line, heterosis for yield was observed due to slightly branched inflorescences in the heterozygous line that increase fruit set in the hybrid compared to its parents. A similar case of epistasis in tomato was discovered in relation to the heterotic mutation single flower truss (sft) and its interaction with the self-pruning gene that belongs to the same family of CET factors (Krieger, U. et al., 2010. Nature genetics. 42, 459-463; WO 2010/041190). Plants heterozygous for loss of function mutations in the sft, the florigen gene, produce 50% higher yield compared to the non-mutant isogenic cultivar M82 and the homozygous sft mutant. The heterotic effect on yield was only detected in the determinate genetic background, which is homozygous for the self-pruning (sp) mutation (sft/+; sp−/sp−) while in indeterminate tomatoes (Sp+/_) there was no effect of sft/+ on yield indicating that Sp+nullifies the heterotic effect of its family member sft. Contrary to these described cases, where epistasis was unintentionally discovered, the inventors of the present invention has devised systematic methods to discover favorable epistatic interactions in a genome wide manner.

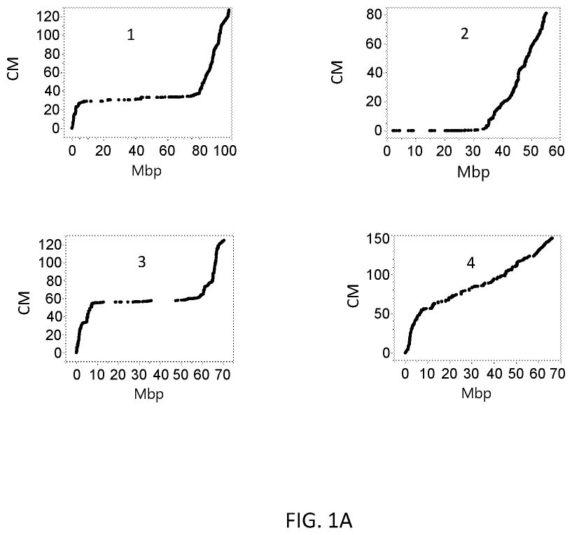

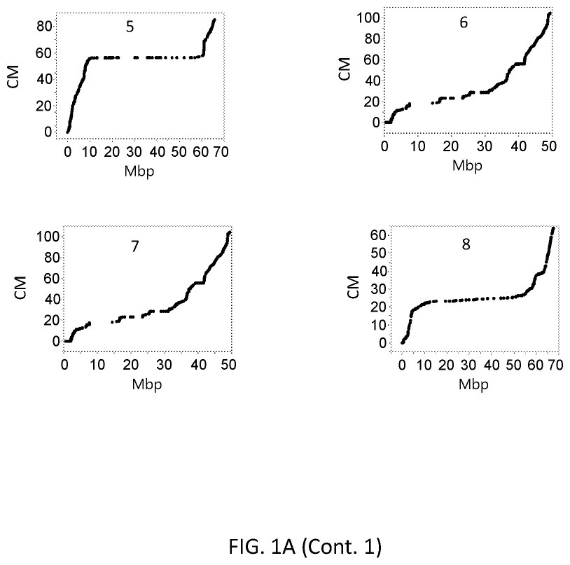

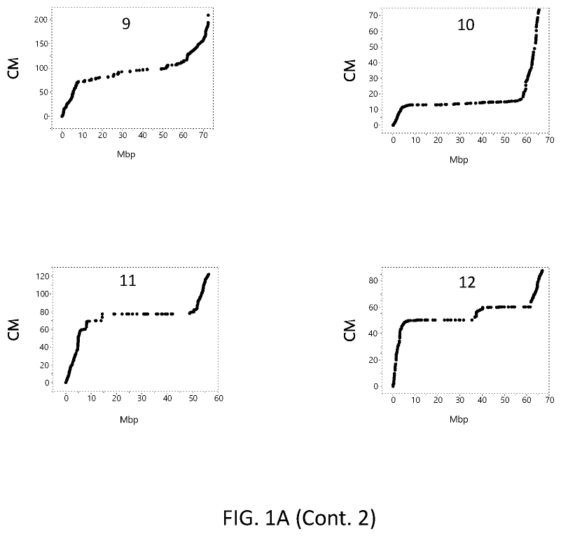

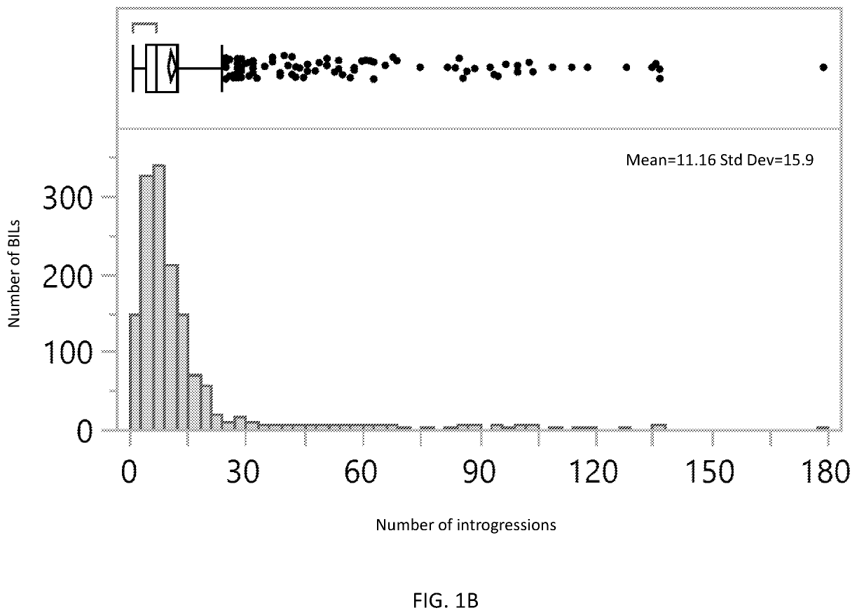

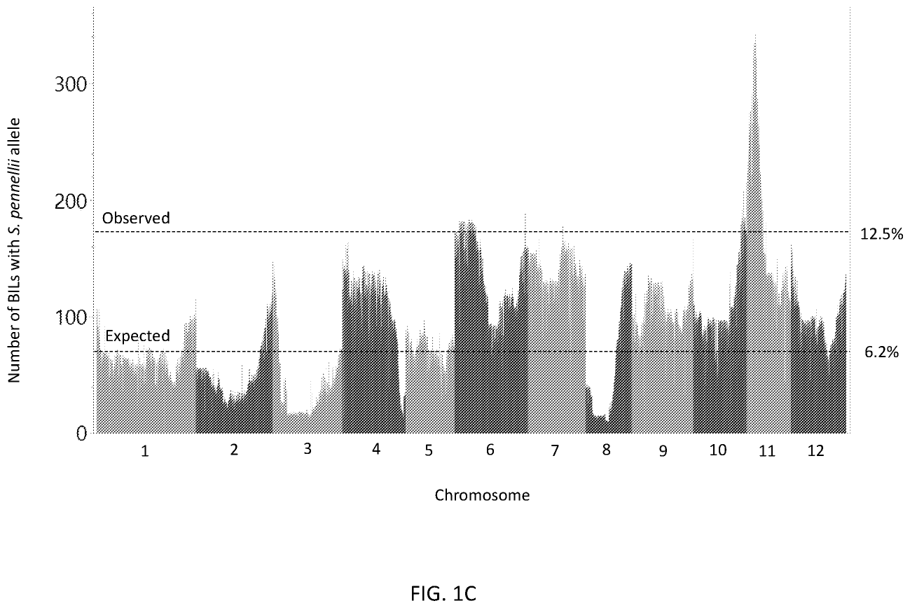

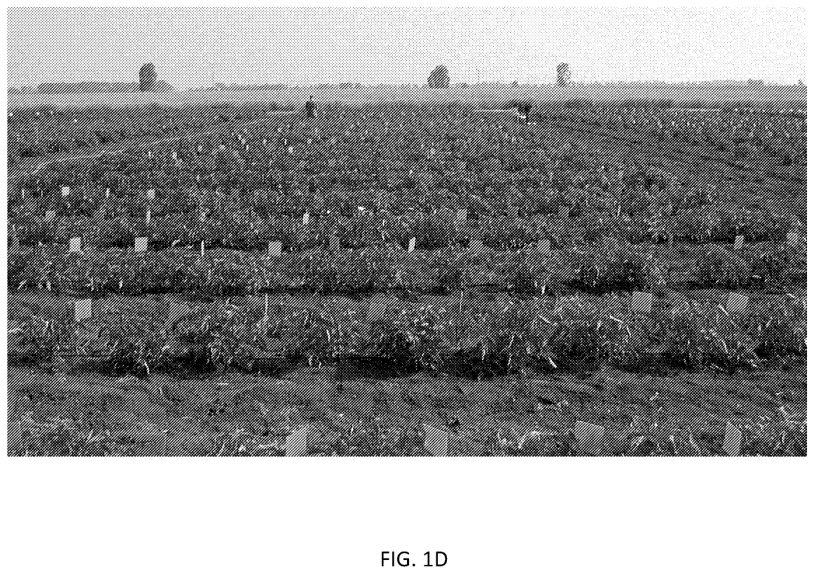

The first factor that limits the mapping of epistatic interactions in segregating populations is the number of individuals. For this the inventors of the present invention developed a very large, highly polymorphic permanent mapping population of backcross inbred lines (BILs) from a cross of a divergent wild species (S. pennellii; the Lost Accession LA5240) and a processing tomato inbred (LEA). The population was composed of 1400 plants in BC2S6 generation, and it was expected that it would be large enough to analyze whole genome digenic epistasis for yield associated traits. Unexpectedly, many of the wild species' alleles deviated from the expected Mendelian segregations (Zamir and Tadmor, 1986, ibid) causing the proportion of plants that carry the S. pennellii alleles to be lower than expected (6% compared to 12.5%; FIG. 1D). For this reason, the analysis of epistasis was conducted only for digenic combinations where the double introgression genotype included at least ten individuals. When the phenotypes of the homozygous BILs containing an average 11 introgressions per BIL were analyzed, their yield was 50% lower than the LEA control. However, in the BILHs (BILxLEA) the mean yield was doubled, indicating that the best use of this resource is for breeding of hybrids. A second limitation for the mapping of epistatic interactions results from the ‘multiple testing penalty’ of all pairs of genomic regions (in the case of digenic interactions), which causes a strong downward adjustment of the statistical significance threshold. The BILs dataset included 7699 SPET markers and thus close to 60×10⁶ digenic combinations whose testing could lead to many false positives. An important advantage of the BILs is that they provide the means for quick and simple validation of candidate cases of epistasis and thus eliminate the need to use very stringent statistical thresholds in the epistasis discovery phase. The validation is done by crossing the double introgression hybrid to LEA or other tomato inbred lines and planting ˜100 plants whose genotypes are expected to be 1_1, 2_1, 1_2 and 2_2 in roughly equal numbers. Genotyping and phenotyping of these plants provides a quick and easy validation protocol that does not suffer from the multiple testing penalty which can eliminate favorable combinations that can be of value for the development of elite crop plants.

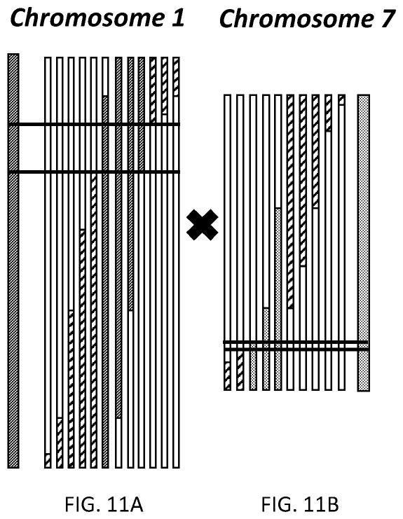

The BILs were found to include a recombinant every 18 Kb in the euchromatin, which is equivalent to a recombinant between every tomato gene. Since the majority of the 80 cases of epistasis that were detected for the yield related traits involved genomic regions in the euchromatin, the BILs also provide the means for fine mapping of the QTL involved by virtue of the high number of mapped recombinants. For fine mapping of the epistatic QTL on chromosome 1 and 7, the two BILs that create the epistasis and the mapped recombinant BILs for each of the chromosomes can be used (FIG. 11). The BILH for the chromosome 1 QTL (dark gray chromosome, FIG. 11A) are crossed to homozygous BILs that are recombinant in chromosome 7. The progeny of such crosses would produce nearly isogenic hybrids of two genotypes: with or without the S. pennellii QTL on chromosome 1. The yield of the isogenic hybrids is compared and if the hybrids with the chromosome 1 QTL would have a higher yield, then the assumption is that the recombined segment of the BIL on chromosome 7 carries the second QTL needed to generate heterosis. A comparison of the values of the two genotypic groups using multiple recombinants BILs would indicate the location of the chromosome 7 QTL. To map the QTL on chromosome 1 which is involved in the interaction, the BILH of the chromosome 7 QTL (light gray chromosome FIG. 11B) are crossed to recombinants BILs of chromosome 1 and the scheme described above is followed to fine map the genomic region of the QTL on chromosome 1.

It is well recognized in genetics that when a gene or a QTL is introduced into different genetic backgrounds the phenotypic outcome may vary, indicating epistatic interactions with unknown factor/s in the receptor genomes. The role of epistasis is also well known in hybrid breeding where often hundreds of homozygous inbred lines are crossed to create many experimental F1 hybrids which are evaluated for yield heterosis and other traits. In processing tomatoes in California approximately 10,000 new hybrids are tested every year and roughly only ten of them make it to the commercial market. Thus, only few F1 hybrids show competitive heterosis indicating that very specific interactions between the parents' genomes are needed to produce a top yielding variety. Using the teachings of the present invention, very large, controlled populations can be developed, providing the means to identify the genomic components that amplify heterosis.

The present invention discloses markers that indicate the presence of the QTL of the invention within a genome of a tomato plant. It is to be noted that the QTL markers disclosed herein are non-limiting. Additional markers may further be identified. In general, the location of a QTL is indicated by a contiguous string of markers that exhibit statistical correlation to the phenotypic trait. Once a marker is found outside that string (i.e., one that has a LOD-score below a certain threshold, indicating that the marker is so remote that recombination in the region between that marker and the QTL occurs so frequently that the presence of the marker does not correlate in a statistically significant manner to the presence of the phenotype) the boundaries of the QTL are set. Thus, it is also possible to indicate the location of the QTL by other markers located within that specified region.

According to additional embodiments of the invention, the QTL markers disclosed herein can also be used to indicate the presence of the QTL (and thus of the phenotype) in an individual plant, i.e., they can be used in marker assisted selection (MAS) procedures. In principle, the number of potentially useful markers is limited, but a large number of markers can be also used. The skilled person may easily identify additional markers to those disclosed in the present application. Any marker that is linked to the QTL, e.g., falling within the physically boundaries of the genomic region spanned by the markers having established LOD scores above a certain threshold thereby indicating that no or very little recombination between the marker and the QTL occurs in crosses; as well as any marker in linkage disequilibrium to the QTL may be used in MAS procedures. Accordingly, the markers identified in the present invention as associated to the QTL, including the marker SSL2.50CH01_95261222 and SSL2.50CH07_65737800, are mere examples of markers suitable for use in MAS procedures.

According to certain aspects, the present invention provides a method for identifying and selecting a tomato plant characterized by an increased crop productivity, comprising the steps of:
a. providing a plurality of tomato crop plants;
b. examining a nucleic acid sample obtained from each of the plurality of the tomato crop plant for the presence of a Chr-1 QTL and Chr-7 QTL, thereby identifying a population comprising both QTLs; and
c. examining the population comprising both QTLs for enhanced crop productivity compared to a control plant or to a pre-determined productivity score value;
thereby selecting a tomato plant characterized by an increased crop productivity compared to the control plant.

According to certain embodiments, the control plant is a tomato plant comprising none or one of the QTLs.

Introducing the genetic element comprising the epistatic QTL combination can be performed by any method as is known to a person skilled in the art. It is to be explicitly understood that in the recipient tomato (S. lycopersicum) plant produced, the segment comprising the QTL is not in its natural background.

The QTLs of the present invention or the genetic element comprising same may be transferred to a recipient plant by any method as is known to a person skilled in the art.

According to certain embodiments, each of the QTLs can be introduced by crossing a QTL donor plant with a recipient tomato (S. lycopersicum) plant (i.e., by introgression).

As used herein, the terms “introgression” “introgressed” and “introgressing” refer to the translocation of a desired allele(s) (forms of a given gene, genetic determinant or sequences) from a genetic background of one species, variety or cultivar into the genome of another species, variety or cultivar. In one method, the desired allele(s) can be introgressed through a sexual cross between two parents, wherein one of the parents has the desired allele in its genome. The desired allele can include desired gene or genes, a marker locus, a QTL or the like.

According to additional or alternative embodiments, isolated nucleic acid sequence comprising each or a combination of the QTLs can be introduced by genetic engineering means, including transformation or gene editing.

According to certain aspects, the present invention provides an isolated polynucleotide comprising a nucleic acid sequence having at least 96%, identity to the nucleic acid sequence of S. pennellii chromosome 1 between positions 100.789953 cM and 127.0420104 cM.

According to certain embodiments, the isolated polynucleotide comprises a nucleic acid sequence having at least 97%, at least 97.5%, at least 98%, at least 98.5%, at least 99%, at least 99.5%, or more, identity to the nucleic acid sequence of S. pennellii chromosome 1 between positions 100.789953 cM and 127.0420104 cM. According to certain embodiments, the isolated polynucleotide comprises the nucleic acid sequence of S. pennellii Lost Accession LA 5240 chromosome 1 between positions 100.789953 cM and 127.0420104 cM. According to certain embodiments, the segment is starting at position 92541344 bp and ending at position 98424021 bp of said chromosome 1.

According to certain embodiments, the polynucleotide comprises a nucleic acid sequence having at least 96%, at least 97%, at least 97.5%, at least 98%, at least 98.5%, at least 99%, at least 99.5%, or more, identity to a nucleic acid sequence selected from the group consisting of SEQ ID NO:1, SEQ ID NO:2 and a combination thereof. According to certain embodiments, the polynucleotide comprises the nucleic acid sequence set forth in the nucleic acid sequence selected from the group consisting of SEQ ID NO: 1, SEQ ID NO:2 and a combination thereof.

According to certain exemplary embodiments, the isolated polynucleotide comprises at least one marker amplified by a pair of primers comprising the nucleic acid sequence set forth in SEQ ID NO:3 and SEQ ID NO:4, wherein the marker is not amenable to digestion by EcoRI. According to certain embodiments, the marker comprises SEQ ID NO:5.

According to certain additional aspects, the present invention provides an isolated polynucleotide comprising a nucleic acid sequence having at least 96% identity to the nucleic acid sequence of S. pennellii chromosome 7 between positions 53.3003069 cM and 56.96038414 cM.

According to certain embodiments, the isolated polynucleotide comprises a nucleic acid sequence having at least 97%, at least 97.5%, at least 98%, at least 98.5%, at least 99%, at least 99.5%, or more, identity to the nucleic acid sequence of S. pennellii chromosome 7 between positions 53.3003069 cM and 56.96038414 cM. According to certain embodiments, the isolated polynucleotide comprises the nucleic acid sequence of S. pennellii Lost Accession LA 5240 chromosome7 between positions 53.3003069 cM and 56.96038414 cM.

According to certain embodiments, the segment is starting at position 66067020 bp and ending at position 66509915 bp of said chromosome 7.

According to certain embodiments, the polynucleotide comprises a nucleic acid sequence having at least 96%, at least 97%, at least 97.5%, at least 98%, at least 98.5%, at least 99%, at least 99.5%, or more, identity to a nucleic acid sequence selected from the group consisting of SEQ ID NO:6, SEQ ID NO:7 and a combination thereof. According to certain embodiments, the polynucleotide comprises the nucleic acid sequence selected from the group consisting of SEQ ID NO:6, SEQ ID NO:7 and a combination thereof.

According to certain exemplary embodiments, the isolated polynucleotide comprises at least one marker amplified by a pair of primers comprising the nucleic acid sequence set forth in SEQ ID NO:8 and SEQ ID NO:9, wherein the marker is not amenable to digestion by HphI. According to certain embodiments, the marker comprises SEQ ID NO:10.

According to additional aspects, the present invention provides a nucleic acid construct comprising at least one isolated polynucleotide of the invention, further comprising at least one regulatory element for directing transcription of the nucleic acid sequence in the tomato host plant cell.

According to certain embodiments, the regulatory element is selected from the group consisting of an enhancer, a promoter, a translation termination sequence, and the like. According to some embodiments of the invention, the regulatory sequence is operably linked to the isolated polynucleotide.

A nucleic acid sequence (particularly a coding nucleic acid sequence) is “operably linked” to a regulatory sequence (e.g., promoter) if the regulatory sequence is capable of exerting a regulatory effect on the coding sequence linked thereto.

According to certain embodiments, the nucleic acid construct is an expression vector comprising a promoter operably linked to Chr-1 QTL and/or Chr-7 QTL of the invention as are described herein.

As used herein, the term “promoter” refers to a region of DNA placed upstream of the transcriptional initiation site of a polynucleotide to which RNA polymerase binds to initiate transcription of RNA. The promoter controls where (e.g., which portion of a plant) and/or when (e.g., at which stage or condition in the lifetime of an organism or a cell thereof) the gene is expressed.

According to some embodiments of the invention, the promoter is heterologous to the isolated polynucleotide and/or to the host cell.

As used herein the phrase “heterologous promoter” refers to a promoter from a different species or from the same species but from a different gene locus as of the isolated polynucleotide sequence.

Any suitable promoter sequence can be used within the nucleic acid construct of the present invention. Preferably the promoter is selected from the group consisting of a constitutive promoter, a tissue-specific, or a developmental-stage specific promoter.

The nucleic acid construct of the present invention can further comprise at least one marker (reporter) gene, operably linked to a regulatory element (such as a promoter) that allows transformed cells containing the marker to be either recovered by negative selection (by inhibiting the growth of cells that do not contain the selectable marker gene), or by positive selection (by screening for the product encoded by the markers gene). Many commonly used selectable marker genes for plant transformation are known in the art, and include, for example, genes that code for enzymes that metabolically detoxify a selective chemical agent which may be an antibiotic or an herbicide, or genes that encode an altered target which is insensitive to the inhibitor. Several positive selection methods are known in the art, such as mannose selection. Alternatively, marker-less transformation can be used to obtain plants without mentioned marker genes, the techniques for which are known in the art.

The construct according to the present invention being a transformation vector, an expression vector or a combination thereof can be, for example, plasmid, a bacmid, a phagemid, a cosmid, a phage, a virus, or an artificial chromosome.

The polynucleotides of the invention and construct comprising same can be chemically synthesized by any method as is known in the Art.

Methods for transforming a plant cell with nucleic acids sequences according to the present invention are known in the art. As used herein the term “transformation” or “transforming” describes a process by which a foreign nucleic acid sequence, such as a vector, enters and changes a recipient cell into a transformed, genetically modified or transgenic cell. Transformation may be stable, wherein the nucleic acid sequence is integrated into the plant genome and as such represents a stable and inherited trait, or transient, wherein the nucleic acid sequence is expressed by the cell transformed but is not integrated into the genome, and as such represents a transient trait. According to typical embodiments the nucleic acid sequences of the present invention are stably transformed into a plant cell.

There are various methods of introducing foreign nucleic acid sequences into both monocotyledonous and dicotyledonous plants (for example, Potrykus I. 1991. Annu Rev Plant Physiol Plant Mol Biol 42:205-225; Shimamoto K. et al., 1989. Nature 338:274-276).

The principal methods of the stable integration of exogenous DNA into plant genomic DNA includes two main approaches:

Agrobacterium-mediated gene transfer: The Agrobacterium-mediated system includes the use of plasmid vectors that contain defined DNA segments which integrate into the plant genomic DNA. Methods of inoculation of the plant tissue vary depending upon the plant species and the Agrobacterium delivery system. A widely used approach is the leaf-disc procedure, which can be performed with any tissue explant that provides a good source for initiation of whole-plant differentiation (Horsch et al., 1988. Plant Molecular Biology Manual A5, 1-9, Kluwer Academic Publishers, Dordrecht).

Direct nucleic acid transfer: There are various methods of direct nucleic acid transfer into plant cells. In electroporation, protoplasts are briefly exposed to a strong electric field, opening up mini-pores to allow DNA to enter. In microinjection, the nucleic acid is mechanically injected directly into the cells using micropipettes. In microparticle bombardment, the nucleic acid is adsorbed on microprojectiles such as magnesium sulfate crystals or tungsten particles, and the microprojectiles are physically accelerated into cells or plant tissues. Another method for introducing nucleic acids to plants is via the sonication of target cells. Alternatively, liposome or spheroplast fusion has been used to introduce expression vectors into plants.

Following transformation of tomato target tissues, expression of the above described selectable marker genes allows for preferential selection of transformed cells, tissues and/or plants, using regeneration and selection methods now well known in the art.

According to other embodiments, the QTLs of the present invention or the at least one nucleic acid construct comprising same can be introduced into the genome of a receptor tomato plant using the techniques of genome editing. These techniques are particularly useful for introducing the QTLs into a pre-determined location within the genome of the recipient tomato plant.

Genome editing is a reverse genetics method which uses artificially engineered nucleases to cut and create specific double-stranded breaks at a desired location(s) in the genome, which are then repaired by cellular endogenous processes such as, homology directed repair (HDR) and non-homologous end-joining (NHEJ). NHEJ directly joins the DNA ends in a double-stranded break, while HDR utilizes a homologous sequence as a template for regenerating the missing DNA sequence at the break point. In order to introduce specific nucleotide modifications to the genomic DNA, a DNA repair template containing the desired sequence must be present during HDR. Genome editing cannot be performed using traditional restriction endonucleases since most restriction enzymes recognize a few base pairs on the DNA as their target and the probability is very high that the recognized base pair combination will be found in many locations across the genome resulting in multiple cuts not limited to a desired location. To overcome this challenge and create site-specific single- or double-stranded breaks, several distinct classes of nucleases have been discovered and bioengineered to date. These include the meganucleases, Zinc finger nucleases (ZFNs), transcription-activator like effector nucleases (TALENs) and CRISPR/Cas system.

According to certain embodiments, the present invention provides a tomato of the species Solanum lycopersicum crop plant genetically engineered to comprise within its genome at least one exogenous nucleic acid construct comprising a QTL derived from chromosome 1 (Chr-1 QTL) and a QTL derived from chromosome 7 (Chr-7 QTL) of Solanum pennellii Lost Accession LA5240, wherein each of the Chr-1 QTL and the Chr-7 QTL is present in the tomato plant in a heterozygous form and wherein the combination of QTLs confers an increase in the crop plant productivity compared to the productivity of a corresponding crop plant comprising none or one QTL.

According to certain embodiments, the QTLs or the genetic element(s) comprising same is introduced to a plurality of plants.

According to certain embodiments, introducing the QTLs or the genetic element(s) comprising same is followed by crossing and selection of offspring plants comprising the QTL and exhibiting the epistatic yield enhancement. A population of plants that are transformed with the cloned Chr-1 QTL and Chr-7 QTL form population heterozygous for the QTLs. Upon self-crossing of the heterozygous plants, the offspring are examined for the QTLs presence using the markers disclosed herein. Selected homozygous plants are used as pollen donors to other female plants not harboring the QTL, producing the desired population of heterozygous to the epistatic QTLs.

The following examples are presented in order to more fully illustrate some embodiments of the invention. They should, in no way be construed, however, as limiting the broad scope of the invention. One skilled in the art can readily devise many variations and modifications of the principles disclosed herein without departing from the scope of the invention.

### EXAMPLES

### Materials and Methods

### Plant Material

In the year 2007 we started to develop a new BIL population for genetic analysis of epistasis. The donor wild species for this resource was an unexplored accession of S. pennellii that was discovered in the Gatersleben collection (Lost Accession; LA5240; available at C. M. Rick Tomato Genetics Resource Center). This accession which is self-compatible was crossed to the modern determinate processing tomato inbred called LEA (FIG. 1A). A single F1 hybrid was crossed as a male to LEA to produce a backcross1 (BC1) of 2000 plants which were crossed again to LEA to produce 2000 BC2 plants. Selfing of the BC2 was carried out until backcross-2-self-6 (BC2S6). Throughout the BIL selfing we planted 2-6 plants from each BIL and extracted seed from a random fertile plant. In BC2S6 we observed close phenotypic resemblance between the siblings of a particular BIL, indicating that the semi-industrial project of pollination, fruit harvest, seed extraction and plantings was carried out accurately. In the more advanced selfing generation we encountered higher sterility which resulted in the production of 1400 BILs.

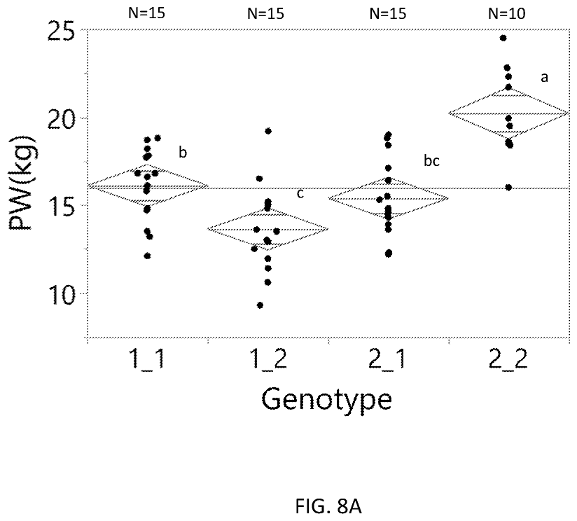

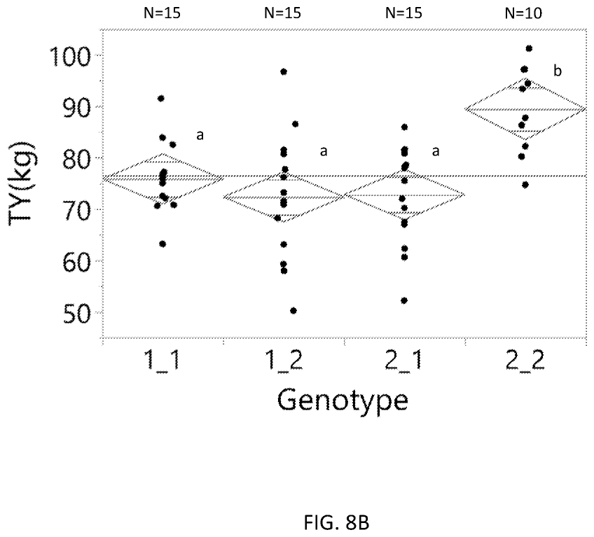

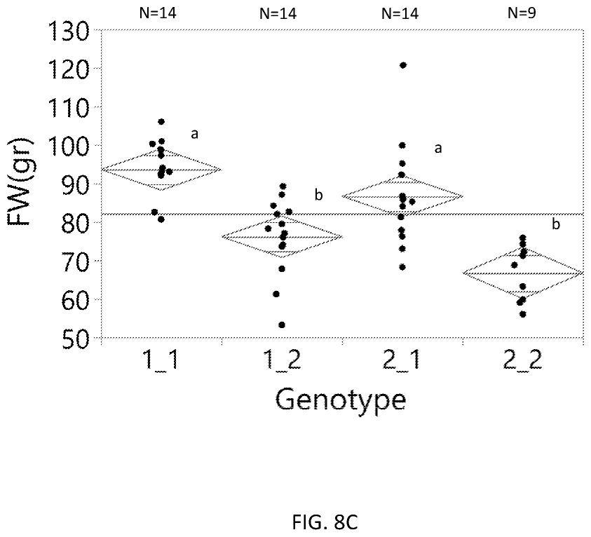

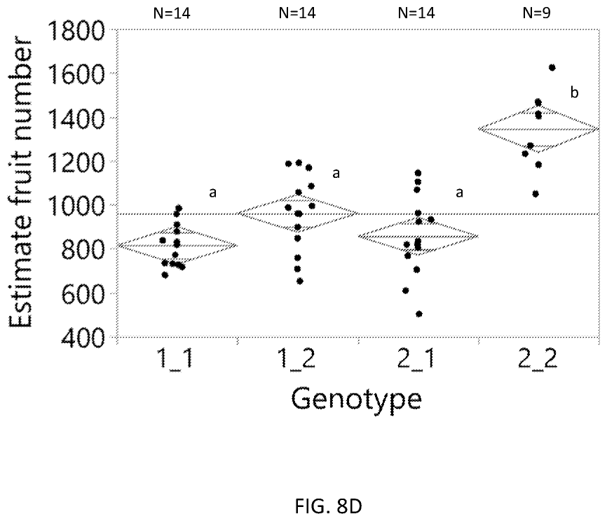

In the fall of 2017, all the 1400 BILs were grown in a greenhouse and were crossed to LEA to create a set of BIL hybrids (BILHs). In the Spring of 2018 seedlings were grown in a greenhouse for 35-40 days and then 1389 BILs, 1233 BILHs and the controls LEA and M82 were transplanted in a drip irrigated field in Akko, Israel. The planting density was 1 plant per m² where the distance between the plants was 0.5 meter and the width of the bed was 2 meters (FIG. 8). Both field plots of “dry” and “wet” treatments started the growing season at field capacity, which represents the maximum amount of water that the soil could hold. For the dry treatments only 60 m³ of water was applied per 1,000 m² of field immediately after transplanting. In the wet treatment, 320 m³ of water was applied per 1,000 m² of field throughout the growing season according to the irrigation protocols in the area. Three weeks after planting the irrigation of the dry treatments was stopped and the plants grew under drought condition with ˜20% of the amount of water that is given to at the normal crop conditions. In the Spring of 2022, we established an experiment where the yield epistasis combinations were tested in a commercial stand in replicated plots of 5 m² with 12 plants per plot.

### Genotyping

Leaflets of each of the 1389 BILs plant grown in Akko field were collected. DNA was extracted using the CTAB protocol and was diluted to a final concentration of 40-60 ng in a volume of 40 μl. DNA quantity and quality were determined using a Nanodrop ND-1000 spectrophotometer followed by electrophoresis on a 1% agarose gel. DNA quantity was validated using Qubit dsDNA BR Assay Kit (Life Technologies, Eugene, OR USA). The BILs DNA and that of the controls LA5240, LEA and M82 was genotyped by Single Primer Enriched Technology (SPET, Barchi et al., 2019, Frontiers in plant science. 10, 1005) using the HiSeq2000 platform by IGA Technology Services, Udine, Italy. A total of 173,000 SNPs were called and subjected to filtering using TASSEL v5.2.43: sites with a depth of less than three reads or >50% missing data were filtered out as well as heterozygous markers, non-polymorphic markers, and markers with minor allele frequency of <1%. The final SNP set included 7699 markers across the 1389 homozygous BILs. Introgressions bins and genetic distances were calculated using Asmap package in R software; (cran.rproject.org/web/packages/ASMap/index.html).

For experimental validation of the epistatic interactions of chromosome 1 and 7 we designed primers for plant genotyping using the site snapgene.com software for alignment of the S. pennellii genome sequence and detection of the polymorphisms in the target regions.

Primers sequence for the chromosome 1 (Chr-1) QTL:

Forward primer:
(SEQ ID NO: 3)
CTATCACTGAAGCAACTAGTGAGG
Reverse primer:
(SEQ ID NO: 4)
CGTTGTTGGTGAATATGAGCTTCAC

The PCR product was of 729 bp and was digested with EcoRI: The S. pennellii allele was not cut while the LEA allele generated two fragments: 503 bp and 226 bp.

Primers sequence for the chromosome 7 (Chr-7) QTL:

Forward primer:
(SEQ ID NO: 8)
ATGGATCGATCGGCTCTGATAC
Reverse primer:
(SEQ ID NO: 9)
GGTAGTCAAAGTTTGACCGACCTT.

The PCR product was 528 bp and was digested with HphI: The S. pennellii allele was not cut while the LEA allele generated two fragments: 354 bp and 174 bp.

### Phenotyping

Fruit of all the genotypes were harvested when 95-100% of the tomatoes were red (105-115 days after transplanting). The following measurements were taken for each of the plants: Plant weight (PW, kg) of the vegetative part, average fruit weight (FW, g) calculated from a random sample of 10 red fruit per plants, total soluble solids concentration (Brix) of the fruit (Bx %) assayed on the same 10 red fruits, and total fresh fruit yield per plant (TY) (including both red and green fruit if any).

### Data Analysis:

Single QTL mapping analysis and two-dimensional genome scans for the detection for epistasis was performed using R/qtl (Broman and Saunak, 2009, ibid) by the Haley-Knott regression. In specific cases single marker effects were reanalyzed by ANOVA using the JMP pro 16 software package (SAS Institute, Cary, NC, USA).

Multiple comparison corrections to significance thresholds were performed for each of the measured phenotypes using 1000 permutations that generated the maximum LOD. The thresholds were set to the 95th percentile (P value<0.05) from the obtained distributions.

### Example 1: Genomic Composition of the Biparental BILs

In 2018 the BC2S6 BILs was genotyped using the single primer enrichment technology (SPET) (Barchi, L, et al. 2019, ibid). Out of the 173,000 SNPs that were scored 7699 SNPs were used, after filtering. The recombination frequencies between the markers were calculated and the position of each marker on the genetic (centi-Morgan, cM) and physical maps (Mbp) of the 12 tomato chromosomes was plotted (FIG. 1A). The chromosome plots show a classic distribution that can be seen in many organisms, in which in the euchromatic regions there are high recombination rates for a physical distance (an average of one recombinant every 18 Kb) whereas in the heterochromatic region there are few recombination events (one recombinant every 700 Kb). It is noteworthy that along the different chromosomes in the heterochromatic regions there are multiple gaps that represent deletions in the S. pennellii genome. The markers that map to the gap regions, all showed homozygosity for the LEA alleles. Similarly, the interspecific F1 hybrid also showed only the LEA alleles in the gap regions, the largest of which is on chromosome 3 (11 Mbp; FIG. 1A). Since the SPET marker design was based on SNPs of the red fruited species it was assumed that had S. pennellii based SPET design was used, many more S. lycopersicum deletions relative to the wild species would have been found, as the genome size of S. pennellii is between 1 to 1.2 Gb compared to 0.9 Gb for the cultivated tomato (Schmidt, Maximilian H-W., et al. 2017. The Plant Cell. 29, 2336-2348). The genotyping analysis of the BILs indicated that an average line carried 11.2 wild species introgressions with a few lines that harbor more than 100 S. pennellii genome segments (FIG. 1B).

The BIL population was designed to allow whole genome two-dimensional QTL scans. The original F1 interspecific hybrid carried the entire genome of the wild species and of the cultivated tomato LEA. In the BC1 and BC2 the average proportion of the wild species genome was reduced by half each generation such that BC2 carried 25% of the wild genome in a heterozygous state. Upon each of the six selfing generations heterozygosity was expected to be reduced by 50% and thus in the sixth generation of selfing every introgression is expected to be present in a homozygous state in 12.5% of the BILs. Consequently, out of the 1400 BILs, 21 BILs are expected to carry two random independent introgressions, which is more than necessary for comparison with the plants that carry a single introgression. However, due to segregation distortion against the wild species alleles, which is common in interspecific crosses (Zamir D. and Tadmor Y. 1986. Botanical Gazette. 147, 355-358), in most of the chromosomes the average prevalence of the S. pennellii introgressions was 6% (FIG. 1C).

### Example 2: Identification of Single and Epistatic QTL

Plants of the BILs and the BIL-hybrids (BILHs) were transplanted in heavy soil in field capacity and irrigated with ˜20% of the amount of water usually used in tomato cultivation (FIG. 1D).

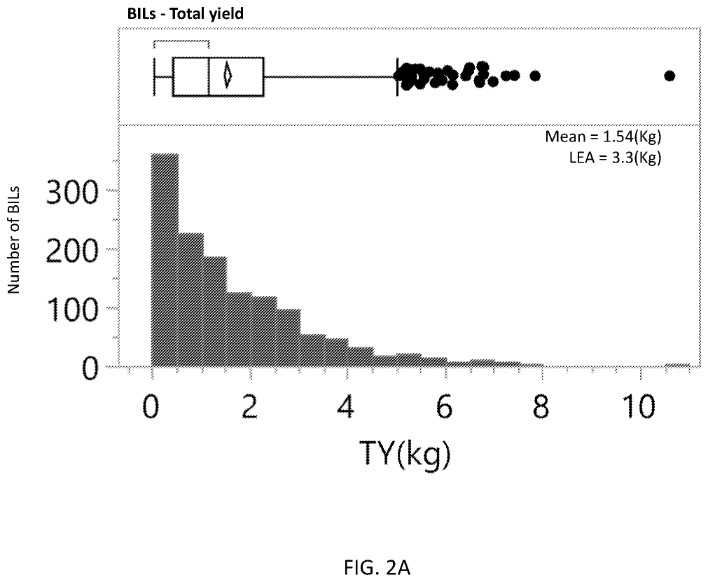

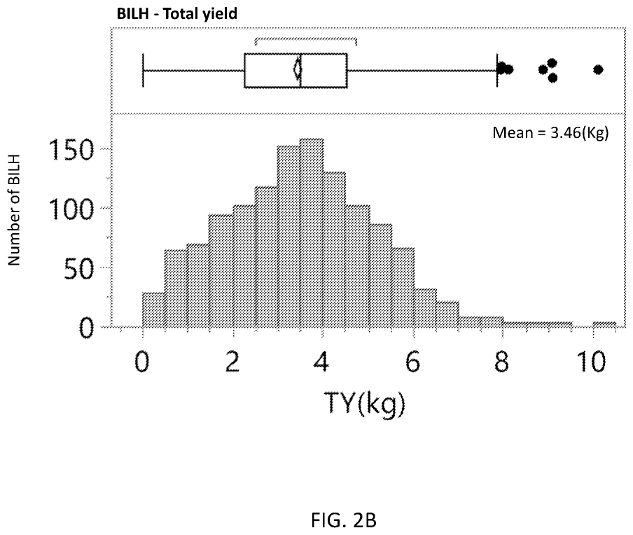

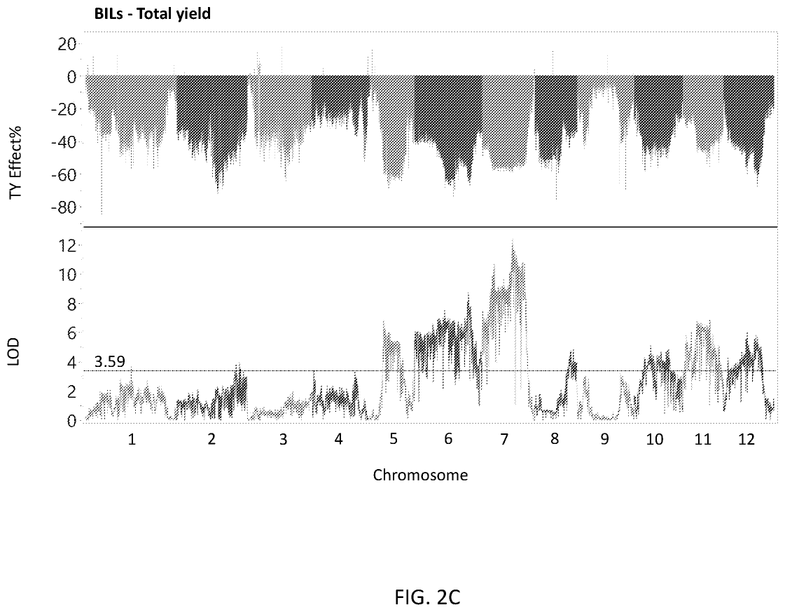

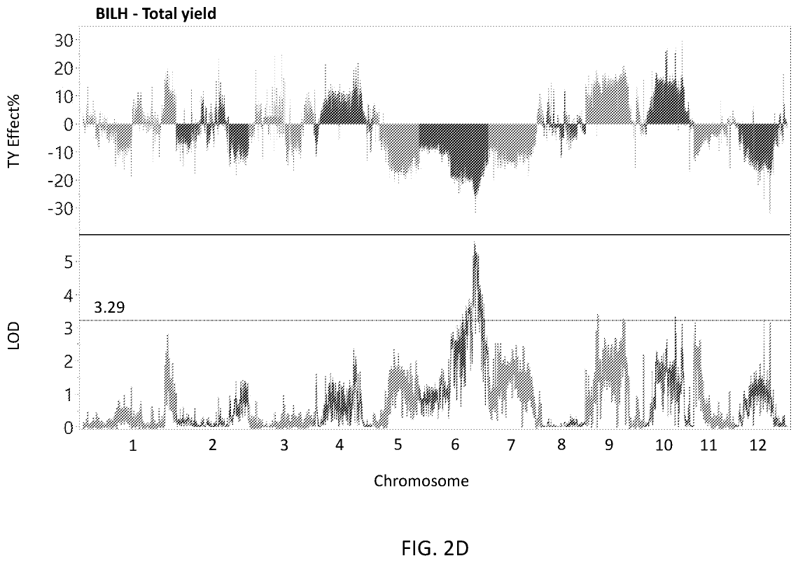

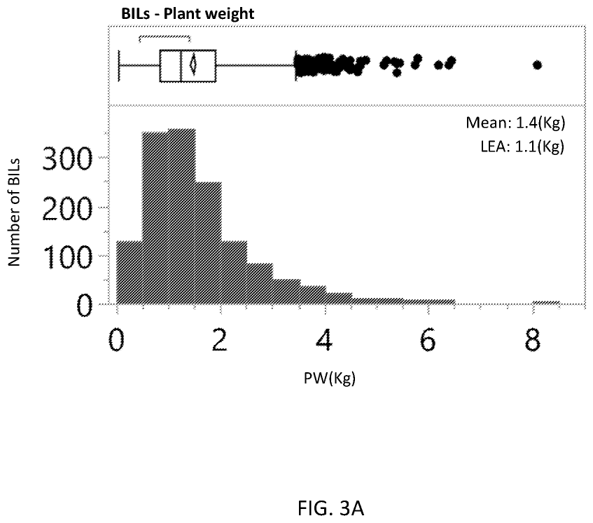

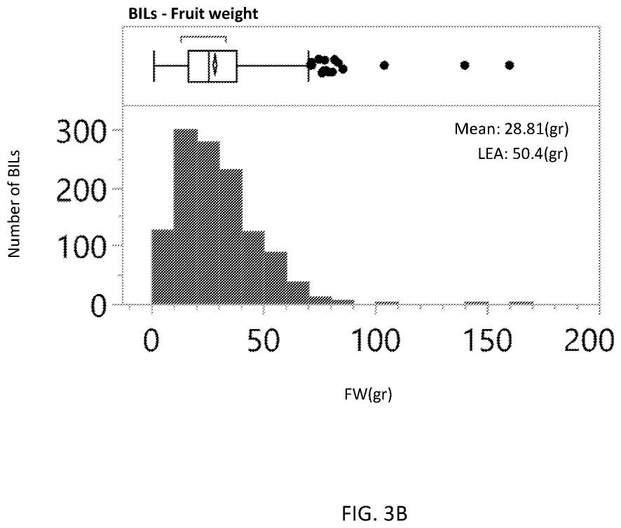

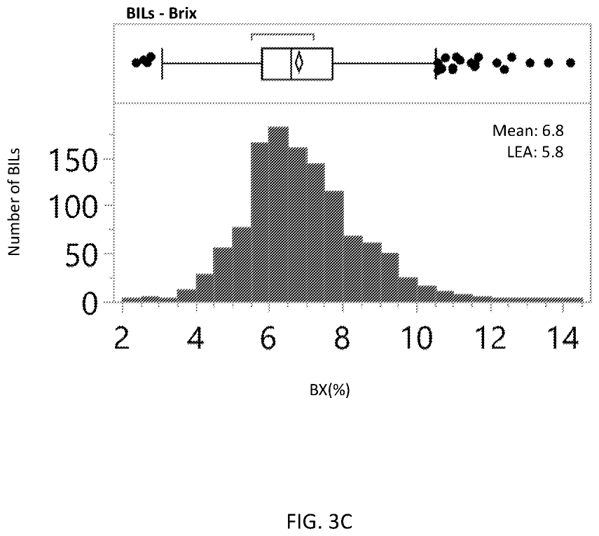

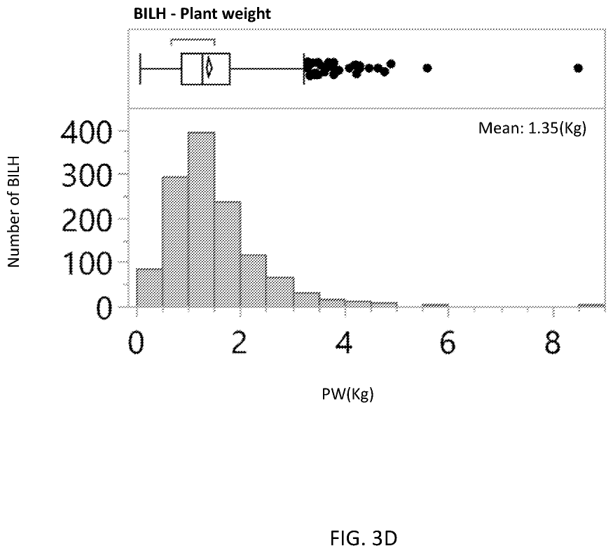

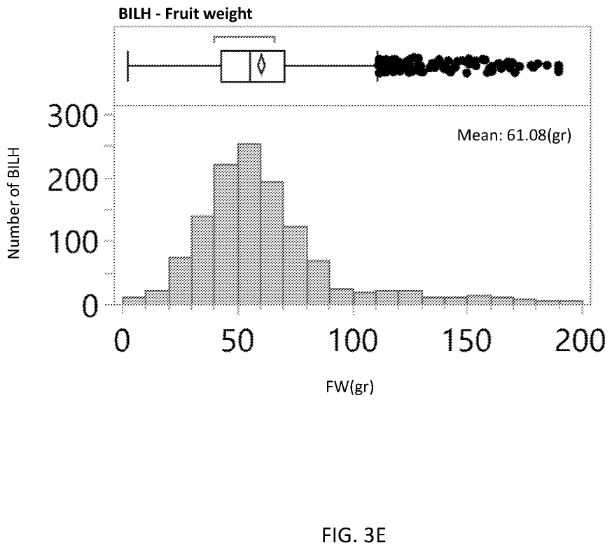

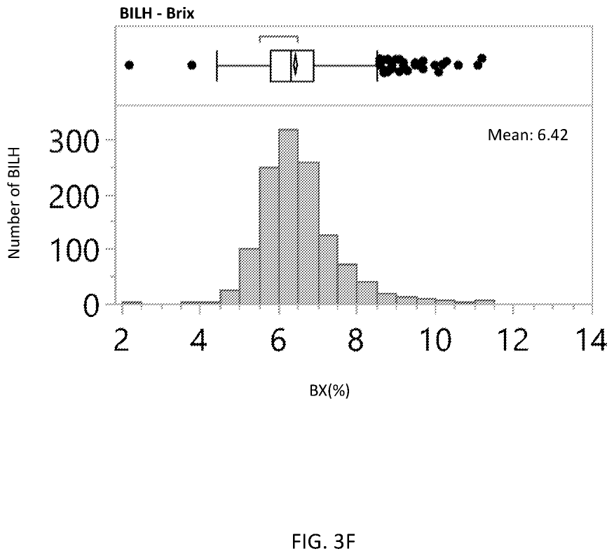

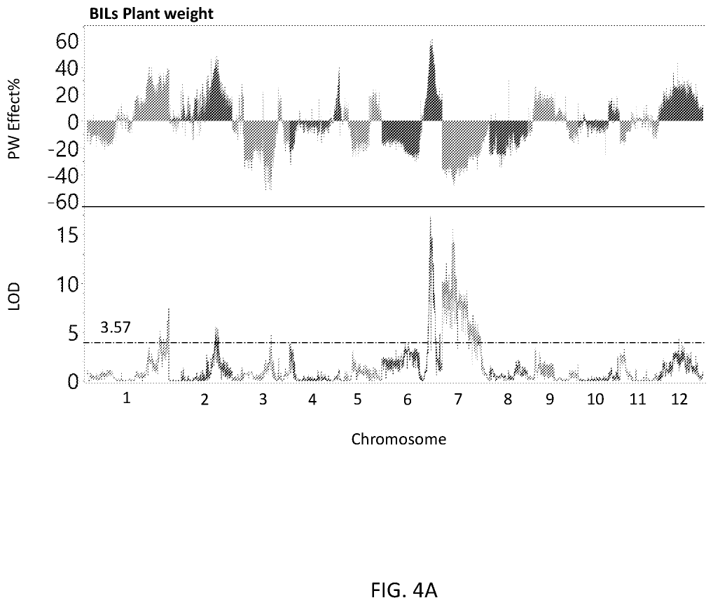

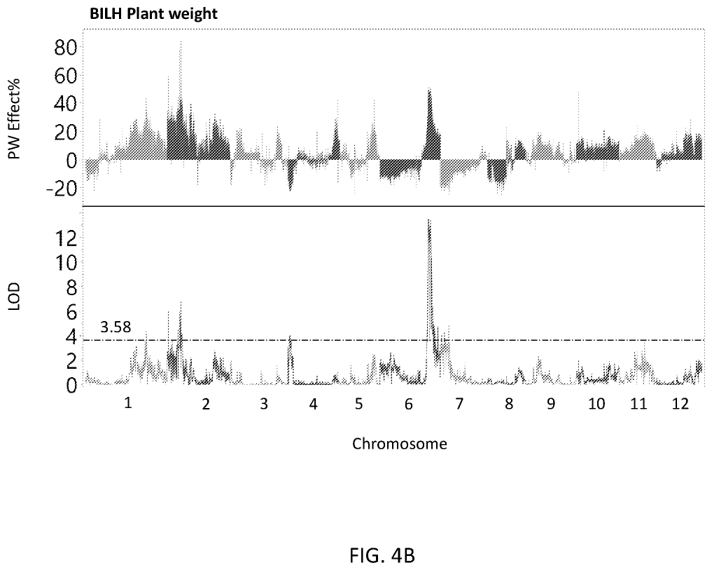

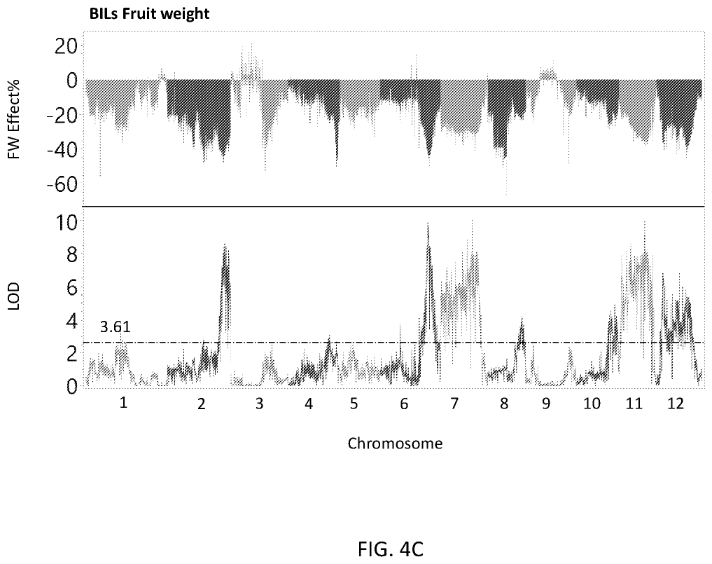

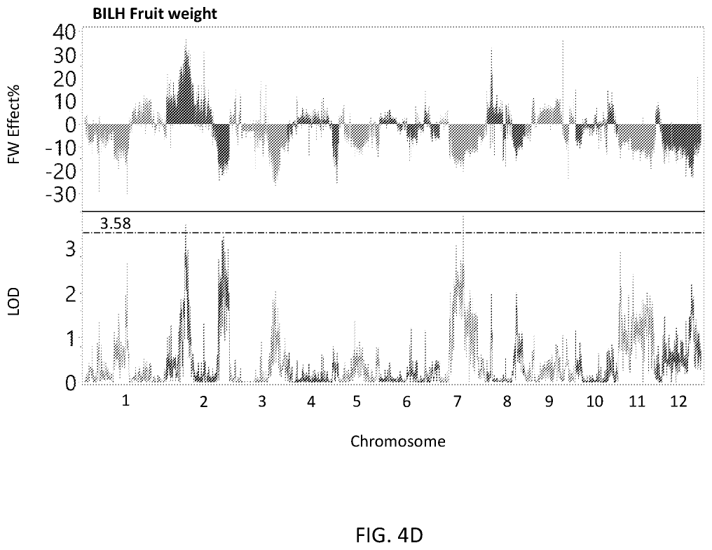

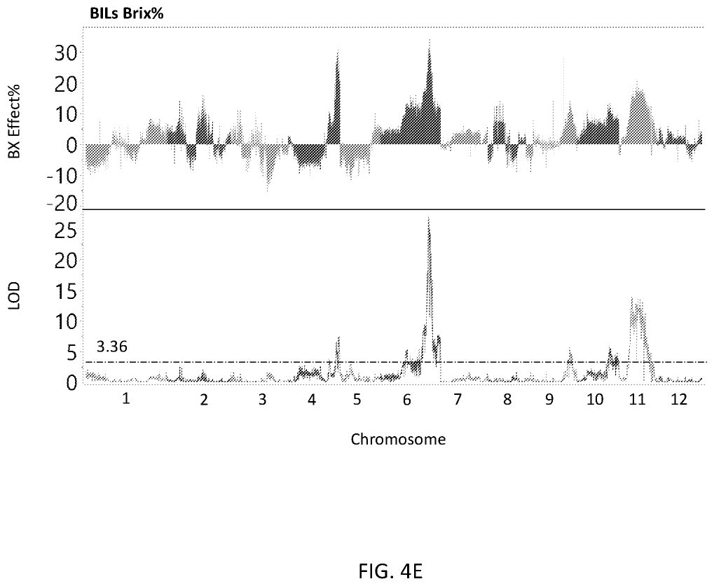

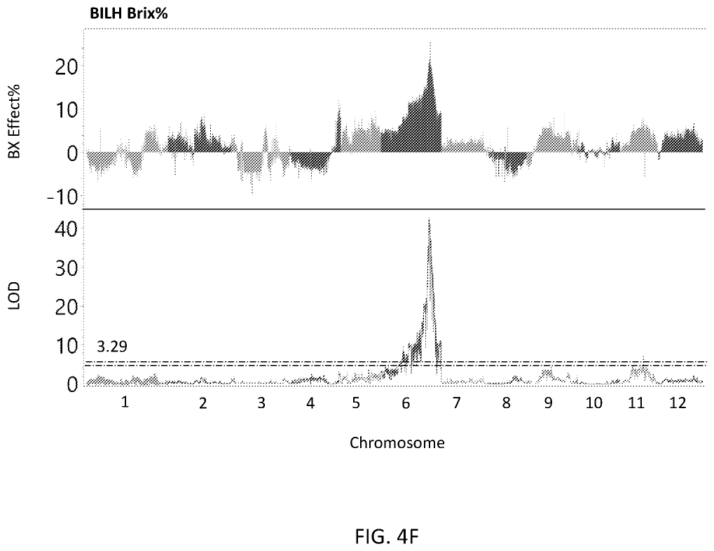

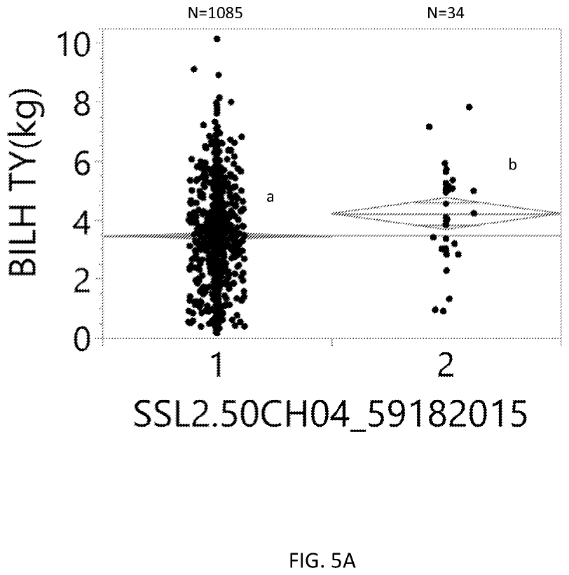

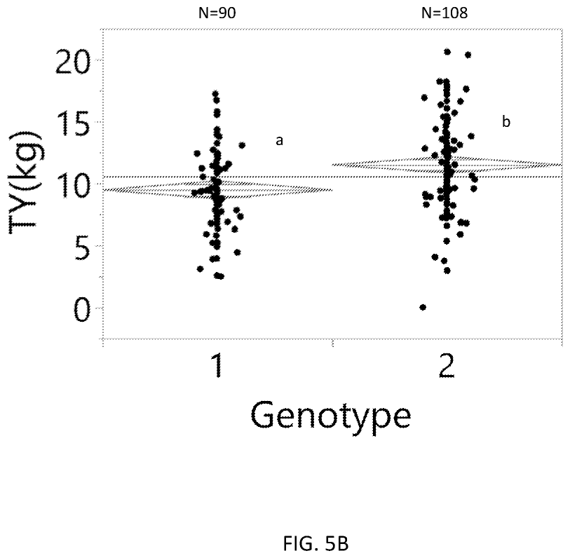

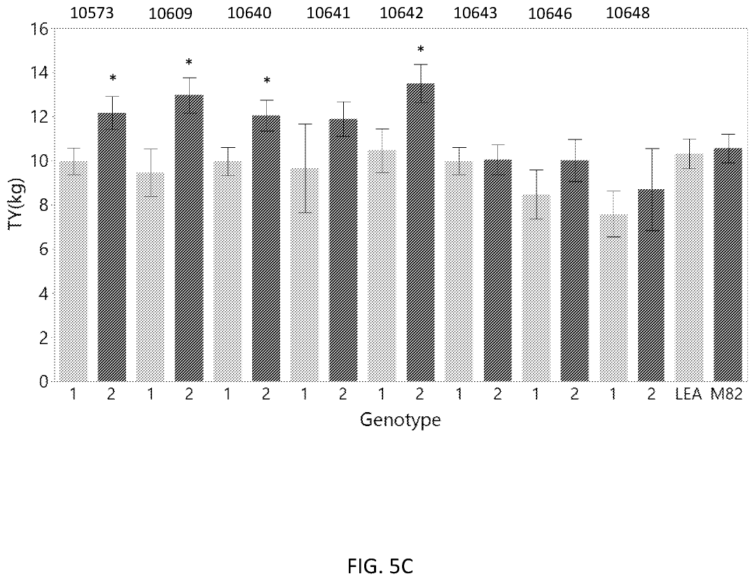

Each plant was phenotyped at harvest time for the following traits: plant weight (PW; only the vegetative part), average fruit weight (FW), Brix (% BX; total soluble solids, mainly sugars) and total yield (TY). As expected, based on previous studies of tomato interspecific crosses, the homozygous BILs produced 50% of the yield relative to the BILHs and LEA (FIGS. 2A and 2B). To improve the productivity of the processing tomato, we focused on breeding F1 hybrids that carry the wild species introgressions in a heterozygous state. This approach is also supported by the single QTL analysis that showed that the BILs introgressions reduced yield relative to the common control LEA by as much as 50%, while in the BILHs, introgressions that significantly increased yield were detected (FIG. 2C and FIG. 2D). The frequency distributions of the other yield-associated traits were close to normal both in the BILs and the BILHs. Mean fruit weight of the BILHs was more than double that of the BILs; the single QTL that affected the measured traits and their significance are indicated (FIG. 3 and FIG. 4). The S. pennellii introgression on chromosome 4 that did not suffer from negative linkage drag, increased yield by 20% relative to the BILH without the introgression (LOD 2.36). This introgression showed in a validation study a 20% yield increase in the LEA background as well as in some additional genetic backgrounds (FIG. 5). This analysis validated the reproducibility of the QTL detected in 2018 and provides the confidence to explore epistasis using the BILs.

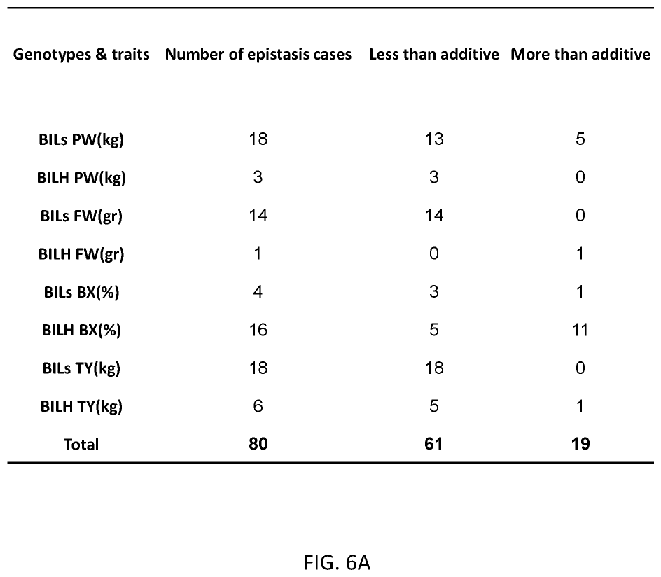

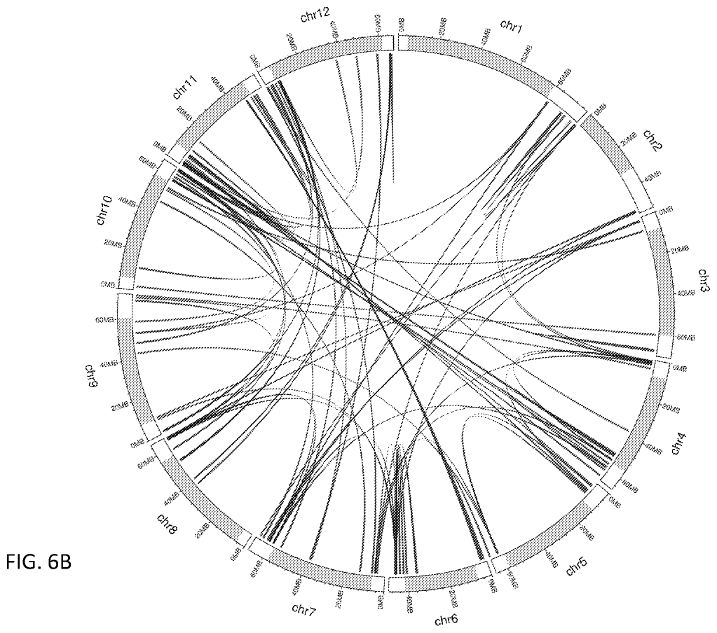

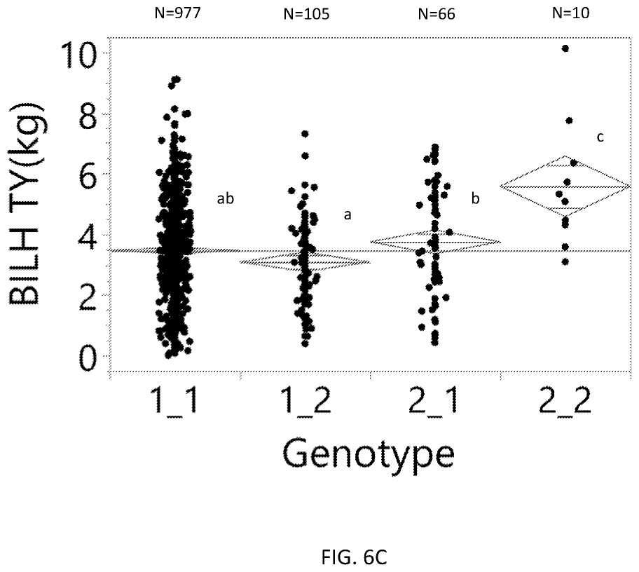

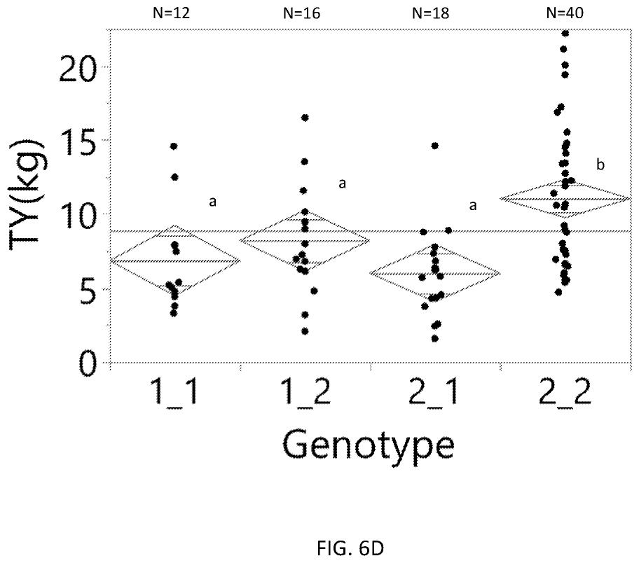

The ability to identify epistasis between two QTL depends on the number of plants that carry the pair of introgressions the phenotype of which is compared to the single QTL hybrids. Due to the deviations from the Mendelian segregations (FIG. 1C), we included in the epistasis analysis only digenic-scans with at least ten plants that carried both QTL alleles from the wild species. A total of 80 epistasis cases were found in the BILs and BILHs, with 61 being less than additive and 19 more than additive (FIG. 6A; Table 1). In the BILs we detected the majority of the less than additive interactions (total 48). For example, for fruit weight and total yield all the homozygous QTL reduced the phenotype relative to the lines that did not carry the introgressions. Based on additivity we would expect the phenotype of the BILs that carry both introgressions to be the sum of two QTL effects. However, in all cases the effect measured on yield and fruit weight in the double introgression BILs was less than the sum of the two QTL. It is important to note that homozygous alleles in the BILs and heterozygous alleles in the BILHs can show epistasis but not necessarily through heterosis. The cases of digenic epistasis were also diagramed on the circus physical map showing that an overwhelming majority of loci involved mapped to the gene rich regions (FIG. 6B). Strikingly, a single epistatic interaction involving introgressions on chromosomes 1 and 7 of S. pennellii, that independently had no effect on yield, when put together, provided for 58% yield increase in the mean of the ten double introgression hybrids (FIG. 6C). To validate this surprising result, selfed seed from the double introgression hybrid were planted and the markers for chromosome 1 (CHR-1, SSL2.50CH01_95261222) and chromosome 7 (CHR-7, SSL2.50CH07_65737800) were examined to select plants from the following four genotypic groups:
1) Plant homozygous for the cultivated tomato alleles in chromosomes 1 and 7 (1_1).
2) Plants heterozygous for the chromosome 1 introgression (2_1).
3) Plants heterozygous for the chromosome 7 introgression (1_2).
4) Plants heterozygous for both introgressions (2_2).

The selected seedlings were planted randomly in an irrigated field where the double heterozygous group (the largest group obtained from the F2) had 37% higher yield than the mean of the three other genotypic groups (FIG. 6D).

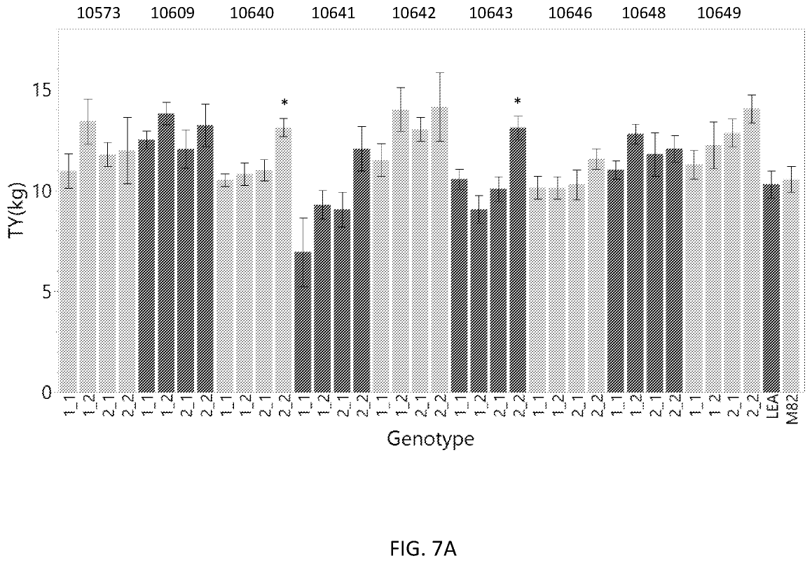

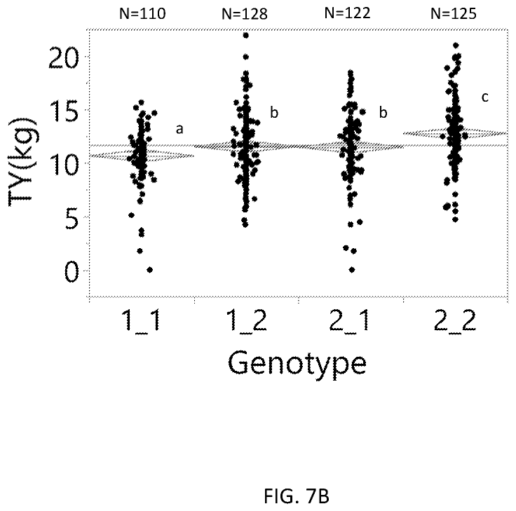

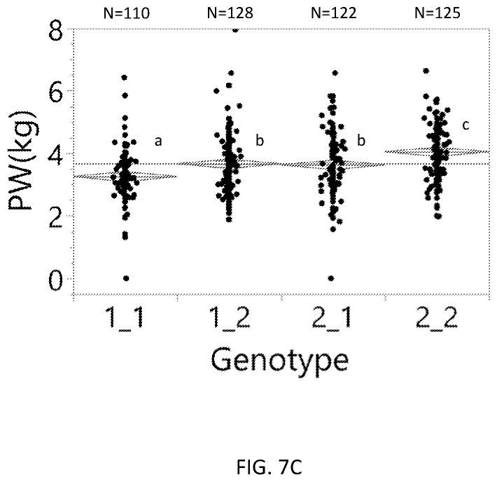

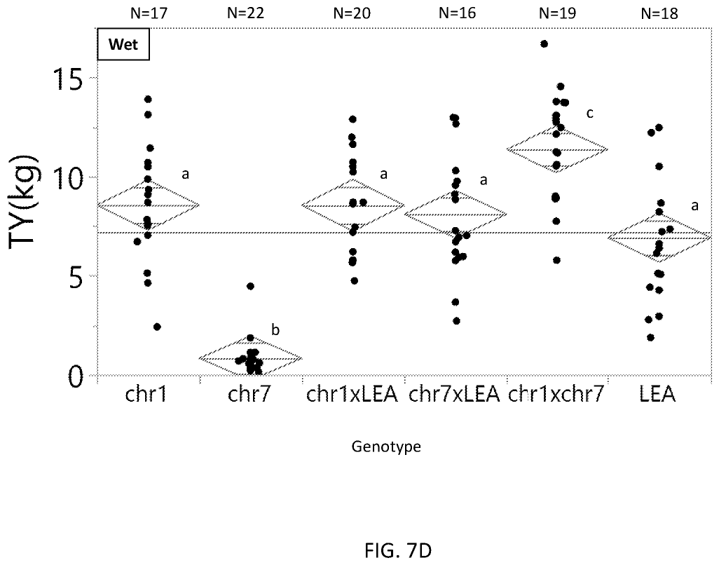

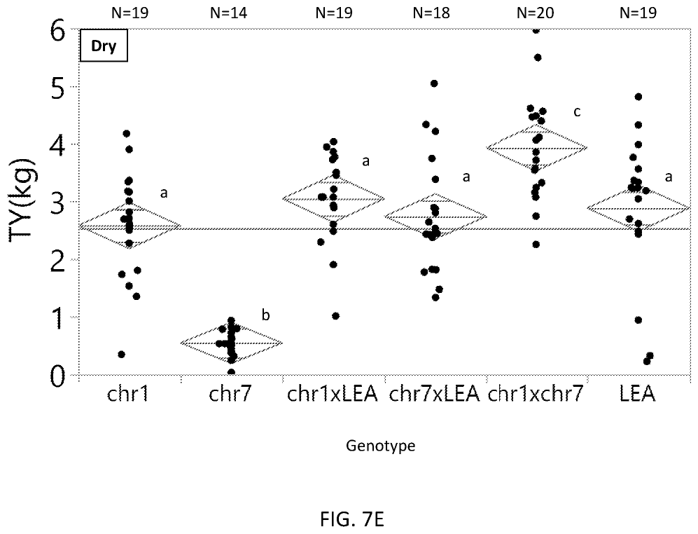

The next question addressed was related to the effect of the genetic background on the epistatic interaction. The double heterozygous LEA hybrid was crossed to nine different processing tomatoes inbred lines and 100 plants from each of the crosses were planted in the field. Genotyping of the plants was done after planting and for each of the families there were between 10 to 15 plants for each of the four genotypic groups (1_1; 2_1; 1_2 and 2_2). The results in FIG. 7A show that in two of the crosses (10640 and 10643) the 2_2 group had a statistically significant higher yield. The average effect of the four genotypic groups over all genetic backgrounds indicates that the double heterozygous group had 13% higher yield (FIG. 7B). The only yield associated trait that correlated with total yield was plant weight (r=0.54) where plants of the 2_2 group had larger vines than the other groups (FIG. 7C).

TABLE 1
Two-dimensional epistasis analysis for 80 significant yield components in the BILs and BILHs

| | | | | First | Second | | | | | | | Effect | 2_1 | 1_2 | | 2_2 - | |
|---|---|---|---|---|---|---|---|---|---|---|---|---|---|---|---|---|---|
| | | | | QTL | QTL | | | | | | Effect | rel- | rel- | rel- | | more/ | |
| | Popu- | | | posi- | posi- | | | | | | rel- | ative | ative | ative | sum | less | |
| | la- | 1st | 2nd | tion | tion | 1_{—} | 2_{—} | 1_{—} | 2_{—} | p. | ative | to best | to | to | QTLs | than | |
| Trait | tion | QTL | QTL | (CM) | (CM) | 1 | 1 | 2 | 2 | value | to LEA | parent | 1_1 | 1_1 | effect | additive | |
| PW | BILs | 1 | 1 | 89.578 | 127.042 | 1.39 | 2.08 | 1.96 | 1.98 | 0.0001 | 0.87 | −0.1 | 0.69 | 0.57 | 1.26 | 0.59 | Less |
| (kg) | | | | | | | | | | | | | | | | | |
| PW | BILs | 1 | 4 | 127.042 | 0.000 | 1.45 | 1.99 | 1.09 | 1.74 | 0.0001 | 0.63 | −0.25 | 0.54 | −0.36 | 0.18 | 0.29 | More |
| (kg) | | | | | | | | | | | | | | | | | |
| PW | BILs | 1 | 7 | 121.029 | 15.981 | 1.47 | 2.09 | 0.74 | 1.14 | 0.0001 | 0.03 | −0.95 | 0.62 | −0.73 | −0.11 | −0.33 | More |
| (kg) | | | | | | | | | | | | | | | | | |
| PW | BILs | 3 | 6 | 16.291 | 80.550 | 1.37 | 1.44 | 2.44 | 1.51 | 0.001 | 0.4 | −0.93 | 0.07 | 1.07 | 1.14 | 0.14 | Less |
| (kg) | | | | | | | | | | | | | | | | | |
| PW | BILs | 4 | 5 | 0.323 | 31.735 | 1.52 | 1.12 | 1.04 | 1.14 | 0.0001 | 0.03 | 0.02 | −0.4 | −0.48 | −0.88 | −0.38 | Less |
| (kg) | | | | | | | | | | | | | | | | | |
| PW | BILs | 5 | 5 | 39.948 | 67.131 | 1.48 | 0.93 | 1.98 | 1.43 | 0.0001 | 0.32 | −0.55 | −0.55 | 0.5 | −0.05 | −0.05 | Less |
| (kg) | | | | | | | | | | | | | | | | | |
| PW | BILs | 5 | 6 | 83.406 | 80.550 | 1.37 | 1.6 | 2.34 | 1.77 | 0.0001 | 0.66 | −0.57 | 0.23 | 0.97 | 1.2 | 0.4 | Less |
| (kg) | | | | | | | | | | | | | | | | | |
| PW | BILs | 6 | 6 | 57.236 | 79.590 | 1.41 | 1.15 | 2.52 | 1.48 | 0.0001 | 0.37 | −1.04 | −0.26 | 1.11 | 0.85 | 0.07 | Less |
| (kg) | | | | | | | | | | | | | | | | | |
| PW | BILs | 6 | 7 | 80.550 | 15.981 | 1.47 | 2.36 | 0.8 | 1.35 | 0.0001 | 0.24 | −1.01 | 0.89 | −0.67 | 0.22 | −0.12 | More |
| (kg) | | | | | | | | | | | | | | | | | |
| PW | BILs | 11 | 4 | 0.000 | 2.956 | 1.57 | 1.13 | 1.27 | 1.4 | 0.0001 | 0.29 | 0.13 | −0.44 | −0.3 | −0.74 | −0.17 | Less |
| (kg) | | | | | | | | | | | | | | | | | |
| PW | BILs | 11 | 5 | 30.558 | 15.253 | 1.54 | 1.06 | 1.27 | 1.65 | 0.0001 | 0.54 | 0.38 | −0.48 | −0.27 | −0.75 | 0.11 | More |
| (kg) | | | | | | | | | | | | | | | | | |
| PW | BILs | 11 | 6 | 80.550 | 18.933 | 1.39 | 2.32 | 1.31 | 2.02 | 0.0001 | 0.91 | −0.3 | 0.93 | −0.08 | 0.85 | 0.63 | Less |
| (kg) | | | | | | | | | | | | | | | | | |
| PW | BILs | 11 | 9 | 27.193 | 2.655 | 1.47 | 1.93 | 1.26 | 1.15 | 0.0001 | 0.04 | −0.78 | 0.46 | −0.21 | 0.25 | −0.32 | More |
| (kg) | | | | | | | | | | | | | | | | | |
| PW | BILs | 11 | 12 | 13.622 | 60.118 | 1.47 | 1.26 | 1.96 | 1.35 | 0.0001 | 0.24 | −0.61 | −0.21 | 0.49 | 0.28 | −0.12 | More |
| (kg) | | | | | | | | | | | | | | | | | |
| PW | BILs | 12 | 4 | 0.000 | 54.689 | 1.47 | 1.09 | 1.87 | 1.86 | 0.0001 | 0.75 | −0.01 | −0.38 | 0.4 | 0.02 | 0.39 | More |
| (kg) | | | | | | | | | | | | | | | | | |
| PW | BILs | 12 | 7 | 15.981 | 54.689 | 1.49 | 0.8 | 1.97 | 1.16 | 0.0001 | 0.05 | −0.81 | −0.69 | 0.48 | −0.21 | −0.33 | More |
| (kg) | | | | | | | | | | | | | | | | | |
| PW | BILs | 12 | 9 | 26.736 | 60.118 | 1.4 | 1.78 | 1.79 | 1.78 | 0.0001 | 0.67 | −0.01 | 0.38 | 0.39 | 0.77 | 0.38 | Less |
| (kg) | | | | | | | | | | | | | | | | | |
| PW | BILs | 12 | 12 | 49.808 | 54.689 | 1.41 | 2.1 | 2.25 | 1.67 | 0.0001 | 0.56 | −0.58 | 0.69 | 0.84 | 1.53 | 0.26 | Less |
| (kg) | | | | | | | | | | | | | | | | | |
| PW | BILH | 4 | 7 | 9.573 | 9.549 | 1.46 | 1.14 | 1.1 | 0.71 | 0.0001 | −0.4 | −0.43 | −0.32 | −0.36 | −0.68 | −0.75 | Less |
| (kg) | | | | | | | | | | | | | | | | | |
| PW | BILH | 6 | 6 | 70.750 | 80.550 | 1.36 | 3.95 | 2.36 | 1.77 | 0.0001 | 0.66 | −2.18 | 2.59 | 1 | 3.59 | 0.41 | Less |
| (kg) | | | | | | | | | | | | | | | | | |
| PW | BILH | 11 | 4 | 2.090 | 90.576 | 1.4 | 1.17 | 1.77 | 1.1 | 0.0001 | −0.01 | −0.67 | −0.23 | 0.37 | 0.14 | −0.3 | Less |
| (kg) | | | | | | | | | | | | | | | | | |
| PW | BILs | 1 | 7 | 39.339 | 37.879 | 30.2 | 21.1 | 19.53 | 14.49 | 0.0001 | −41.62 | −6.61 | −9.1 | −10.67 | −19.77 | −15.71 | Less |
| (kg) | | | | | | | | | | | | | | | | | |
| FW | BILs | 1 | 10 | 39.339 | 52.374 | 30.33 | 20.4 | 22.57 | 20.63 | 0.0001 | −35.48 | −1.94 | −9.93 | −7.76 | −17.69 | −9.7 | Less |
| (gr) | | | | | | | | | | | | | | | | | |
| FW | BILs | 2 | 7 | 72.401 | 37.803 | 30.8 | 19.26 | 20.41 | 12.4 | 0.0001 | −43.71 | −8.01 | −11.54 | −10.39 | −21.93 | −18.4 | Less |
| (gr) | | | | | | | | | | | | | | | | | |
| FW | BILs | 2 | 8 | 68.515 | 35.608 | 30.53 | 16.5 | 23.21 | 18.3 | 0.0001 | −37.81 | −4.91 | −14.03 | −7.32 | −21.35 | −12.23 | Less |
| (gr) | | | | | | | | | | | | | | | | | |
| FW | BILs | 6 | 6 | 55.708 | 77.434 | 29.5 | 32.64 | 17.5 | 14.54 | 0.0001 | −41.57 | −18.1 | 3.14 | −12 | −8.86 | −14.96 | Less |
| (gr) | | | | | | | | | | | | | | | | | |
| FW | BILs | 7 | 8 | 37.803 | 33.426 | 30.55 | 19.58 | 23.57 | 18.04 | 0.0001 | −38.07 | −5.53 | −10.97 | −6.98 | −17.95 | −12.51 | Less |
| (gr) | | | | | | | | | | | | | | | | | |
| FW | BILs | 10 | 2 | 72.401 | 52.374 | 30.83 | 17.18 | 22.7 | 20.58 | 0.0001 | −35.53 | −2.12 | −13.65 | −8.13 | −21.78 | −10.25 | Less |
| (gr) | | | | | | | | | | | | | | | | | |
| FW | BILs | 10 | 4 | 136.347 | 54.907 | 30.01 | 22.86 | 23.06 | 25.9 | 0.0001 | −30.21 | 2.84 | −7.15 | −6.95 | −14.1 | −4.11 | Less |
| (gr) | | | | | | | | | | | | | | | | | |
| FW | BILs | 10 | 7 | 37.803 | 52.374 | 30.85 | 20.07 | 23.4 | 16.7 | 0.0001 | −39.41 | −6.7 | −10.78 | −7.45 | −18.23 | −14.15 | Less |
| (gr) | | | | | | | | | | | | | | | | | |
| FW | BILs | 10 | 8 | 35.608 | 53.931 | 30.69 | 22.6 | 22.86 | 20.09 | 0.0001 | −36.02 | −2.77 | −8.09 | −7.83 | −15.92 | −10.6 | Less |
| (gr) | | | | | | | | | | | | | | | | | |
| FW | BILs | 10 | 11 | 66.853 | 87.021 | 31.6 | 23.79 | 17.8 | 24.1 | 0.0001 | −32.01 | 0.31 | −7.81 | −13.8 | −21.61 | −7.5 | Less |
| (gr) | | | | | | | | | | | | | | | | | |
| FW | BILs | 11 | 7 | 37.803 | 101.921 | 31.1 | 19.55 | 18.7 | 18.62 | 0.0001 | −37.49 | −0.93 | −11.55 | −12.4 | −23.95 | −12.48 | Less |
| (gr) | | | | | | | | | | | | | | | | | |
| FW | BILs | 11 | 8 | 37.106 | 87.021 | 30.9 | 23.9 | 19.59 | 21.6 | 0.0001 | −34.51 | −2.3 | −7 | −11.31 | −18.31 | −9.3 | Less |
| (gr) | | | | | | | | | | | | | | | | | |
| FW | BILs | 12 | 8 | 37.106 | 58.629 | 29.9 | 24.4 | 20.22 | 18.8 | 0.0001 | −37.31 | −5.6 | −5.5 | −9.68 | −15.18 | −11.1 | Less |
| (gr) | | | | | | | | | | | | | | | | | |
| FW | BILH | 4 | 9 | 35.706 | 181.868 | 61.29 | 54.38 | 57.99 | 91.24 | 0.0006 | 35.13 | 33.25 | −6.91 | −3.3 | −10.21 | 29.95 | More |
| (gr) | | | | | | | | | | | | | | | | | |
| Brix | BILs | 3 | 7 | 13.004 | 38.560 | 6.74 | 7.35 | 7.14 | 5.75 | 0.0001 | −0.05 | −1.6 | 0.61 | 0.4 | 1.01 | −0.99 | Less |
| (%) | | | | | | | | | | | | | | | | | |
| Brix | BILs | 4 | 4 | 116.214 | 132.214 | 6.83 | 5.86 | 8.18 | 6.9 | 0.0001 | 1.1 | −1.28 | −0.97 | 1.35 | 0.38 | 0.07 | Less |
| (%) | | | | | | | | | | | | | | | | | |
| Brix | BILs | 10 | 11 | 54.907 | 80.496 | 6.6 | 7.3 | 8.1 | 7.38 | 0.0001 | 1.58 | −0.72 | 0.7 | 1.5 | 2.2 | 0.78 | Less |
| (%) | | | | | | | | | | | | | | | | | |
| Brix | BILs | 11 | 11 | 52.855 | 77.412 | 6.68 | 7.04 | 6.58 | 8.11 | 0.0001 | 2.31 | 1.07 | 0.36 | −0.1 | 0.26 | 1.43 | More |
| (%) | | | | | | | | | | | | | | | | | |
| Brix | BILH | 1 | 1 | 76.651 | 83.791 | 6.4 | 6.35 | 7.67 | 6.4 | 0.001 | 0.6 | −1.27 | −0.05 | 1.27 | 1.22 | 0 | Less |
| (%) | | | | | | | | | | | | | | | | | |
| Brix | BILH | 1 | 4 | 82.538 | 12.651 | 6.44 | 6.31 | 6.31 | 7.5 | 0.0004 | 1.7 | 1.19 | −0.13 | −0.13 | −0.26 | 1.06 | More |
| (%) | | | | | | | | | | | | | | | | | |
| Brix | BILH | 1 | 9 | 39.339 | 105.562 | 6.39 | 6.2 | 6.66 | 7.59 | 0.0001 | 1.79 | 0.93 | −0.19 | 0.27 | 0.08 | 1.2 | More |
| (%) | | | | | | | | | | | | | | | | | |
| Brix | BILH | 3 | 9 | 19.349 | 71.874 | 6.37 | 6.47 | 6.66 | 7.27 | 0.0002 | 1.47 | 0.61 | 0.1 | 0.29 | 0.39 | 0.9 | More |
| (%) | | | | | | | | | | | | | | | | | |
| Brix | BILH | 5 | 5 | 19.782 | 67.131 | 6.4 | 6.48 | 6.56 | 7.72 | 0.0001 | 1.92 | 1.16 | 0.08 | 0.16 | 0.24 | 1.32 | Less |
| (%) | | | | | | | | | | | | | | | | | |
| Brix | BILH | 5 | 6 | 60.491 | 80.550 | 6.32 | 6.3 | 7.4 | 7.8 | 0.0001 | 2 | 0.4 | −0.02 | 1.08 | 1.06 | 1.48 | More |
| (%) | | | | | | | | | | | | | | | | | |
| Brix | BILH | 5 | 9 | 60.491 | 97.359 | 6.36 | 6.65 | 6.59 | 7.4 | 0.0001 | 1.6 | 0.75 | 0.29 | 0.23 | 0.52 | 1.04 | More |
| (%) | | | | | | | | | | | | | | | | | |
| Brix | BILH | 6 | 6 | 37.459 | 80.992 | 6.28 | 6.6 | 7.33 | 8.49 | 0.0001 | 2.69 | 1.16 | 0.32 | 1.05 | 1.37 | 2.21 | More |
| (%) | | | | | | | | | | | | | | | | | |
| Brix | BILH | 6 | 9 | 80.550 | 64.421 | 6.28 | 7.4 | 6.62 | 7.41 | 0.0001 | 1.61 | 0.01 | 1.12 | 0.34 | 1.46 | 1.13 | More |
| (%) | | | | | | | | | | | | | | | | | |
| Brix | BILH | 9 | 9 | 105.562 | 162.840 | 6.37 | 6.65 | 6.46 | 7.36 | 0.0001 | 1.56 | 0.71 | 0.28 | 0.09 | 0.37 | 0.99 | More |
| (%) | | | | | | | | | | | | | | | | | |
| Brix | BILH | 10 | 3 | 19.349 | 25.457 | 6.41 | 6.48 | 6.42 | 7.27 | 0.0055 | 1.47 | 0.79 | 0.07 | 0.01 | 0.08 | 0.86 | More |
| (%) | | | | | | | | | | | | | | | | | |
| Brix | BILH | 10 | 4 | 125.801 | 20.067 | 6.43 | 6.17 | 6.4 | 6.78 | 0.0514 | 0.98 | 0.38 | −0.26 | −0.03 | −0.29 | 0.35 | More |
| (%) | | | | | | | | | | | | | | | | | |
| Brix | BILH | 10 | 6 | 80.550 | 52.686 | 6.3 | 7.58 | 6.49 | 7.02 | 0.0001 | 1.22 | −0.56 | 1.28 | 0.19 | 1.47 | 0.72 | Less |
| (%) | | | | | | | | | | | | | | | | | |
| Brix | BILH | 10 | 9 | 105.562 | 18.171 | 6.37 | 6.65 | 6.4 | 7.24 | 0.0002 | 1.44 | 0.59 | 0.28 | 0.03 | 0.31 | 0.87 | More |
| (%) | | | | | | | | | | | | | | | | | |
| Brix | BILH | 11 | 11 | 78.121 | 82.030 | 6.36 | 6.95 | 7.5 | 6.9 | 0.001 | 1.1 | −0.6 | 0.59 | 1.14 | 1.73 | 0.54 | More |
| (%) | | | | | | | | | | | | | | | | | |
| Brix | BILH | 12 | 12 | 79.404 | 81.247 | 6.4 | 8.96 | 5.95 | 6.57 | 0.0001 | 0.77 | −2.39 | 2.56 | −0.45 | 2.11 | 0.17 | Less |
| (%) | | | | | | | | | | | | | | | | | |
| TY | BILs | 3 | 3 | 113.830 | 117.058 | 1.48 | 2.77 | 2.4 | 0.8 | 0.0001 | −2.5 | −1.97 | 1.29 | 0.92 | 2.21 | −0.68 | Less |
| (kg) | | | | | | | | | | | | | | | | | |
| TY | BILs | 4 | 7 | 126.681 | 30.564 | 1.67 | 1.05 | 0.68 | 0.77 | 0.0001 | −2.53 | −0.28 | −0.62 | −0.99 | −1.61 | −0.9 | Less |
| (kg) | | | | | | | | | | | | | | | | | |
| TY | BILs | 6 | 6 | 38.004 | 80.550 | 1.66 | 0.92 | 0.56 | 0.43 | 0.0001 | −2.87 | −0.49 | −0.74 | −1.1 | −1.84 | −1.23 | Less |
| (kg) | | | | | | | | | | | | | | | | | |
| TY | BILs | 6 | 7 | 79.590 | 30.564 | 1.7 | 0.62 | 0.7 | 0.47 | 0.0001 | −2.83 | −0.23 | −1.08 | −1 | −2.08 | −1.23 | Less |
| (kg) | | | | | | | | | | | | | | | | | |
| TY | BILs | 6 | 8 | 79.590 | 32.601 | 1.68 | 0.62 | 1 | 0.43 | 0.0001 | −2.87 | −0.57 | −1.06 | −0.68 | −1.74 | −1.25 | Less |
| (kg) | | | | | | | | | | | | | | | | | |
| TY | BILs | 7 | 8 | 30.564 | 35.532 | 1.69 | 0.7 | 1.04 | 0.44 | 0.0001 | −2.86 | −0.6 | −0.99 | −0.65 | −1.64 | −1.25 | Less |
| (kg) | | | | | | | | | | | | | | | | | |
| TY | BILs | 8 | 9 | 35.532 | 153.315 | 1.64 | 0.97 | 0.91 | 0.94 | 0.0001 | −2.36 | −0.03 | −0.67 | −0.73 | −1.4 | −0.7 | Less |
| (kg) | | | | | | | | | | | | | | | | | |
| TY | BILs | 10 | 8 | 32.601 | 18.171 | 1.64 | 1.01 | 0.87 | 0.4 | 0.0001 | −2.9 | −0.61 | −0.63 | −0.77 | −1.4 | −1.24 | Less |
| (kg) | | | | | | | | | | | | | | | | | |
| TY | BILs | 10 | 10 | 13.041 | 54.907 | 1.61 | 1.01 | 1.25 | 0.36 | 0.0001 | −2.94 | −0.89 | −0.6 | −0.36 | −0.96 | −1.25 | Less |
| (kg) | | | | | | | | | | | | | | | | | |
| TY | BILs | 10 | 11 | 18.171 | 58.877 | 1.67 | 0.93 | 1 | 0.23 | 0.0001 | −3.07 | −0.77 | −0.74 | −0.67 | −1.41 | −1.44 | Less |
| (kg) | | | | | | | | | | | | | | | | | |
| TY | BILs | 11 | 4 | 3.906 | 82.649 | 1.67 | 1.09 | 0.72 | 0.67 | 0.0001 | −2.63 | −0.42 | −0.58 | −0.95 | −1.53 | −1 | Less |
| (kg) | | | | | | | | | | | | | | | | | |
| TY | BILs | 11 | 5 | 36.508 | 58.877 | 1.69 | 0.63 | 0.97 | 0.48 | 0.0001 | −2.82 | −0.49 | −1.06 | −0.72 | −1.78 | −1.21 | Less |
| (kg) | | | | | | | | | | | | | | | | | |
| TY | BILs | 11 | 6 | 79.590 | 12.383 | 1.73 | 0.61 | 1.15 | 0.41 | 0.0001 | −2.89 | −0.74 | −1.12 | −0.58 | −1.7 | −1.32 | Less |
| (kg) | | | | | | | | | | | | | | | | | |
| TY | BILs | 11 | 7 | 30.564 | 58.877 | 1.73 | 0.69 | 0.96 | 0.63 | 0.0001 | −2.67 | −0.33 | −1.04 | −0.77 | −1.81 | −1.1 | Less |
| (kg) | | | | | | | | | | | | | | | | | |
| TY | BILs | 11 | 8 | 35.532 | 83.112 | 1.66 | 0.97 | 0.89 | 0.69 | 0.0001 | −2.61 | −0.28 | −0.69 | −0.77 | −1.46 | −0.97 | Less |
| (kg) | | | | | | | | | | | | | | | | | |
| TY | BILs | 11 | 11 | 12.383 | 69.245 | 1.71 | 1.16 | 0.87 | 0.95 | 0.0001 | −2.35 | −0.21 | −0.55 | −0.84 | −1.39 | −0.76 | Less |
| (kg) | | | | | | | | | | | | | | | | | |
| TY | BILs | 12 | 7 | 30.564 | 57.066 | 1.67 | 0.73 | 0.81 | 0.39 | 0.0001 | −2.91 | −0.42 | −0.94 | −0.86 | −1.8 | −1.28 | Less |
| (kg) | | | | | | | | | | | | | | | | | |
| TY | BILs | 12 | 8 | 35.532 | 52.740 | 1.65 | 1.03 | 0.74 | 0.3 | 0.0001 | −3 | −0.73 | −0.62 | −0.91 | −1.53 | −1.35 | Less |
| (kg) | | | | | | | | | | | | | | | | | |
| TY | BILH | 1 | 7 | 114.409 | 50.702 | 3.45 | 3.75 | 3.09 | 5.57 | 0.0001 | 2.27 | 1.82 | 0.3 | −0.36 | −0.06 | 2.12 | More |
| (kg) | | | | | | | | | | | | | | | | | |
| TY | BILH | 4 | 4 | 3.682 | 121.087 | 3.44 | 3.29 | 4.39 | 2.79 | 0.001 | −0.51 | −1.6 | −0.15 | 0.95 | 0.8 | −0.65 | Less |
| (kg) | | | | | | | | | | | | | | | | | |
| TY | BILH | 4 | 7 | 121.087 | 15.981 | 3.46 | 4.28 | 3.04 | 2.73 | 0.001 | −0.57 | −1.55 | 0.82 | −0.42 | 0.4 | −0.73 | Less |
| (kg) | | | | | | | | | | | | | | | | | |
| TY | BILH | 10 | 4 | 125.727 | 16.173 | 3.37 | 3.81 | 3.85 | 4.16 | 0.0039 | 0.86 | 0.31 | 0.44 | 0.48 | 0.92 | 0.79 | Less |
| (kg) | | | | | | | | | | | | | | | | | |
| TY | BILH | 11 | 6 | 80.550 | 3.712 | 3.63 | 2.69 | 3.04 | 2.76 | 0.001 | −0.54 | −0.28 | −0.94 | −0.59 | −1.53 | −0.87 | Less |
| (kg) | | | | | | | | | | | | | | | | | |
| TY | BILH | 11 | 9 | 138.020 | 4.920 | 3.49 | 4.17 | 3 | 2.98 | 0.001 | −0.32 | −1.19 | 0.68 | −0.49 | 0.19 | −0.51 | Less |
| (kg) | | | | | | | | | | | | | | | | | |

### Example 3: Epistatic QTL for Yield Heterosis

To validate the observation that the yield heterosis is driven by digenic epistasis a homozygous BIL for chromosome 1 (p-427) and a BIL for chromosome 7 (p-1573) were used, both covering the QTL locations. The total yield of p-427 was high but p-1573 was partially sterile, likely due to deleterious recessive alleles derived from the wild species. For this reason, both BILs were crossed to LEA to produce single introgression BILH that could be compared phenotypically to the double introgression heterozygotes. The derived genotypes and the LEA control were planted in wet and dry plots since in the initial experiment of 2018 this epistasis improved yield under drought. Plant stand was 1 plant per m². In both treatments the yield of the double introgression hybrid was significantly higher from the inbred LEA and the best heterozygous parent (Chr-1 QTL x LEA; 29% yield improvement under the dry conditions and 33% under the wet conditions; FIG. 7D and FIG. 7E). These results clearly support the conclusion that the observed heterosis is driven by more than additive digenic epistasis.

### Example 4: Further Validation of the QTL Combination Epistatic Effect in the Field

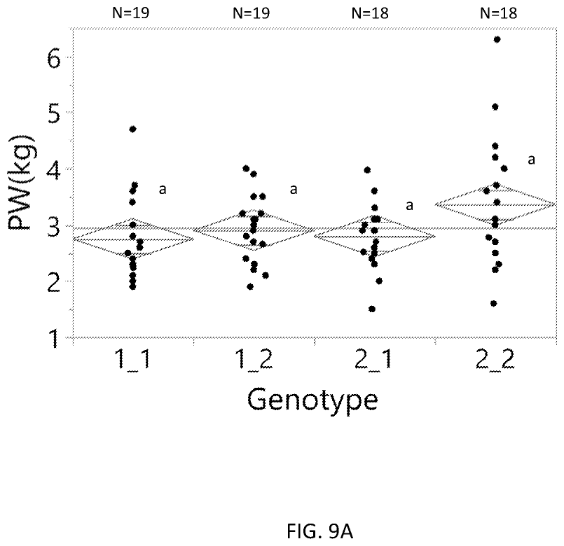

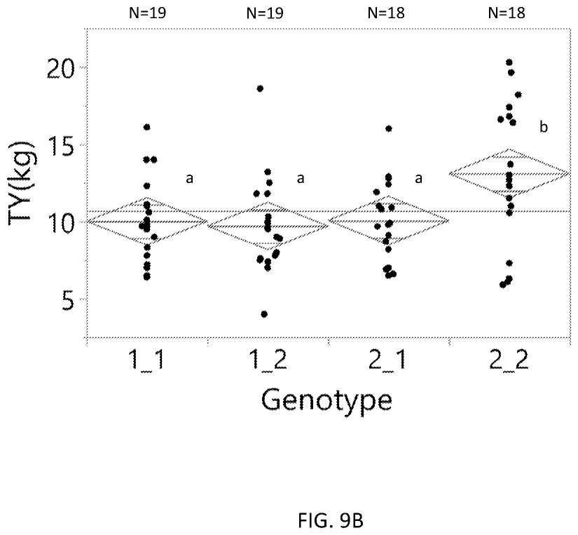

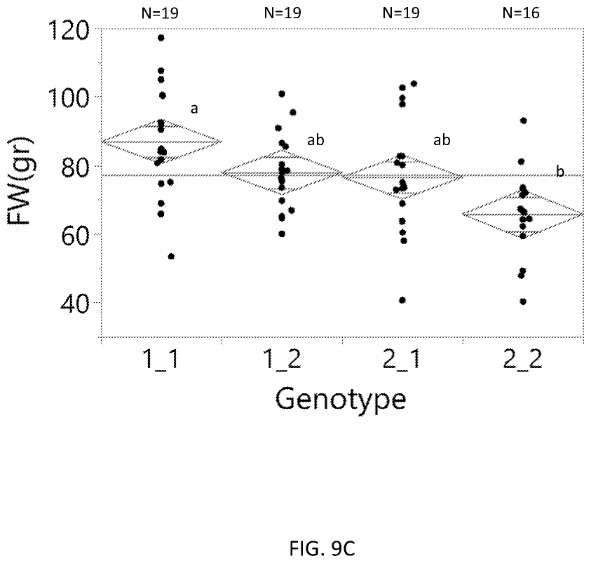

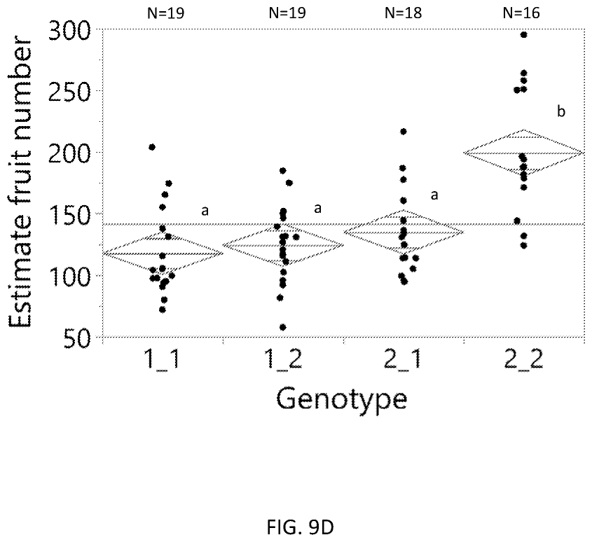

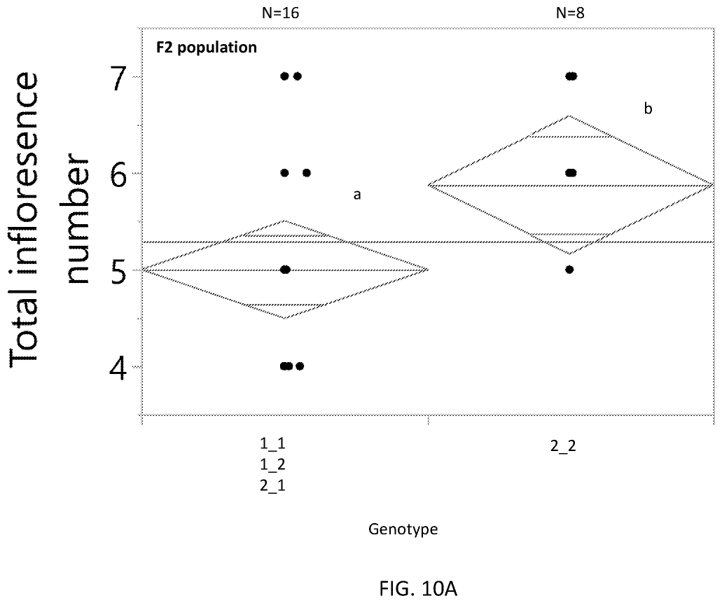

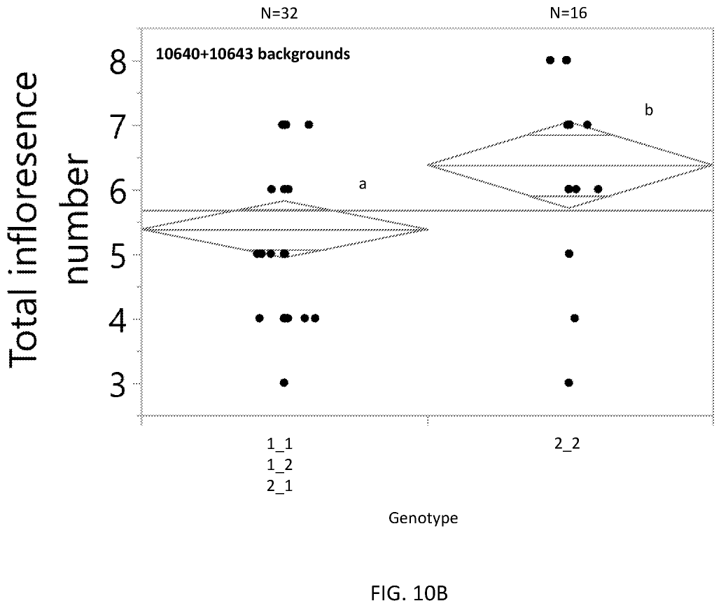

In the experiments described in Example 3 hereinabove, each replication consisted of a single plant grown within 1 m². This planting density is a good way to generate, in an economical way, the many replications that are needed for analysis of complex traits. However, the commercial stand of processing tomatoes is 2.5 plants per m². To evaluate the relevance of the observed heterosis to the tomato crop we conducted an experiment in which each replication consisted of a plot of 5 m² comprising 12 plants with a control experiment with single plant per 1 m². The control experiment yielded similar results compared to the experiments of Example 3. The double heterozygous (2_2) had the largest plant weight, the smallest fruit weight and significantly higher yield (13.1 Kg) compared to the mean of the other three genotypes (1_1, 1_2, 2_1; 10 Kg per plant; FIG. 9). Dividing the total yield by the average fruit weight showed that the double introgression hybrid produced 58% higher number of fruit than the three control genotypes. The plot experiment followed the same trends that were observed in the single plants' experiments. Plant weight for the plots of the double introgression hybrid plots was the highest and their fruit weight was the lowest (FIG. 8A and FIG. 8B). Total yield of the 2_2 plots was 22% higher than the control genotypes and fruit number was 53% higher (FIG. 8C and FIG. 8D). In summary, the reduction of 30% in fruit weight in the double heterozygote genotype was more than compensated for by a large increase in fruit number, which resulted in 22% yield increase in the plots. Both in the single plant and the plot experiments the total yield did not correlate to fruit weight but strongly correlated to plant weight (R² 0.59 and 0.60 respectively (Table 2). To define the cause of the yield heterosis, we planted in the greenhouse plants of the F2 population of the double heterozygote in the LEA background (FIG. 6) and the progeny of its crosses to 10640 and 10643, and counted the number of inflorescences along the main stem (FIG. 10). In both cases the double heterozygotes produced significantly more inflorescences along the main shoot suggesting that an increase in inflorescence number is responsible, at least in part, to the observed yield heterosis.

TABLE 2
Trait correlations in the dense spacing (FIG.
8) and wide spacing (FIG. 9) experiments

| Experiment | Correlation Tests Between traits | r | r² |
|---|---|---|---|
| Dense spacing | Av. Fruit Weight (FW, gr)/Total | −0.17 | 0.03 |
| | Yield (TY, Kg) | | |
| Dense spacing | Plant weight (PW, Kg)/TY (Kg) | 0.77* | 0.6* |
| Dense spacing | PW (Kg)/Av. FW (gr) | −0.1 | 0.01 |
| Wide spacing | Av. FW (gr)/TY (Kg) | 0.2 | 0.04 |
| Wide spacing | PW (Kg)/TY (Kg) | 0.76* | 0.59* |
| Wide spacing | PW (Kg)/Av. FW (gr) | 0.2 | 0.04 |

*P < 0.0001

### Example 5: Validation and Fine-Mapping of the Epistatic QTLs

Plants showing the strongest yield epistasis are selected for validation. It is important to note that when a large number of statistical comparisons is performed, a common scenario in two-dimensional genome scans (or higher), some will have significant P values by chance alone. One way to reduce the number of comparisons is to combine the markers into bins that include co-segregating SNPs or even to combine several very tightly linked consecutive bins into an ‘artificial bin’ (Zhang, W et al., 2016. PLOS Comput Biol 12 (5): e1004925). The Bonferroni correction is a simple way to correct for multiple comparisons. More recently, researchers have been using a number of different procedures to adjust for the false discovery rate (Benjamini, Y and Hochberg Y. 1995. Journal of the Royal Statistical Society, Series B. 57:289-300). The strategy taken in research of the present invention is to employ a mild stringent in screening of the data for candidate epistatic QTL and to implement a simple field validation protocol to eliminate false positives.

The analysis of yield epistasis is conducted using multiple programs that are based on genetic models as well as on those that make no genetic assumptions and employ nonparametric machine learning approaches (www.epistasis.org). Yield data are generated for three blocks of the BILHs with a processing tomato inbred, different from LEA designated 4414, grown under drought conditions. About 20 plants showing the phenotype of “more than additive” type with regard to yield are selected. Additional about 20 plants may be further selected in a consecutive growing season.

The principles of the approach of validation are described herein for a simple case of more than additive epistasis in the BILHs. Seeds of BILHs in the LEA background, which carry both QTLs in the heterozygous state are planted. The resulted plants are crossed to LEA and to 4414. In both crosses the expected progeny comprise 25% of the plants being without the S. pennellii introgression of Chr-1 QTL and Chr-7 QTL; 25% being heterozygous for Chr-1 QTL only, 25% being heterozygous for Chr-7 QTL only and 25% being heterozygous for both QTLs. Progeny of such crosses (100 individuals each) is assayed with PCR markers that flank the introgressions with the QTL and non-recombinant plants are selected and planted in a randomized design in the field under dry growth conditions for validation (25 of each genotype). It is predicted that this cross shows additional segregating S. pennellii introgressions but these introgressions will be randomly distributed in the four genotypic groups and will not affect the analysis.

In a second phase of the analysis of the validated cases, the BILH carrying the entire segment of Chr-1 QTL are crossed to BILs that carry recombinants in the region of Chr-7 QTL. The progeny of each cross produces nearly isogenic lines (NILs) with and without Chr-1 QTL, but both containing an identical recombined Chr-7 QTL segment. If the lines (with the gray-marked colored introgression in FIG. 11B) complement the epistatic phenotype with the NIL that carry Chr-1 QTL (and not with the empty NIL), then the epistatic locus maps between the horizontal lines (FIG. 11). In the reciprocal case, the BILH of Chr-7 QTL is crossed to recombinants of Chr-1 QTL, followed by field phenotyping and selection as above. Plant showing strong epistasis effect of improved productivity are selected for mapping of the underlying genes. Since the recombination rates in the BILs is exceptionally high it allows mapping at least part of the epistatic gene at an open reading frame resolution.

Another dimension of epistasis is explored in the background of additional tomato cultivar TOP. BILs is the interaction between S. pennellii alleles and those of the TOP compared to LEA (the TOP BILs carry genome segments from the three backgrounds). The approach to the validation is similar to the one presented in FIG. 11 since DNA sequence polymorphisms exists between the two cultivated tomato inbred lines.

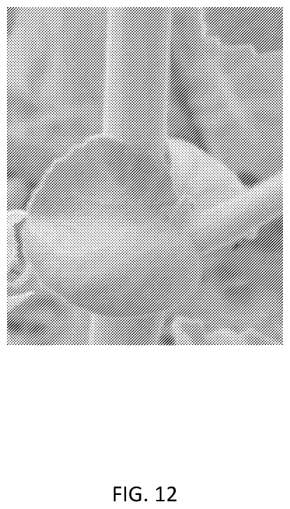

Finally, the simplest case of epistatic interaction is between individual QTL that affect a particular trait in one genetic background but not in another. For example, S. pennellii is characterized by ‘companion leaves’ that are not present in the cultivated tomato (FIG. 12). In the 1500 LEA BILs we found only few plants with companion leaves; however, in the TOP BILs such a phenotype was evident at much higher frequencies. Based on this trait and additional observations made in the greenhouse, multiple cases of QTL genetic background interactions in the LEA and TOP BILs are uncovered. Since the TOP BILs segregate both for S. pennellii introgressions and for LEA introgressions it may be possible to identify and validate the genetic background genes that interact with the wild species introgressions to control an epistatic phenotype. As an example, the indeterminate BILs in the LEA cross had significantly more companion leaves than the determinate ones; and conversely, the determinate BILs in the TOP cross had significantly fewer cases of companion leaves than the indeterminate ones. These results suggest that the background interaction might be associated with Self pruning while demonstrating the power of the population for analysis of epistasis.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

1.-45. (canceled)

46. A tomato crop plant comprising within its genome one or more exogenous genetic elements comprising a quantitative trait locus (QTL) derived from chromosome 1 (Chr-1 QTL) and a QTL derived from chromosome 7 (Chr-7 QTL) of Solanum pennellii Lost Accession LA5240, wherein each of the Chr-1 QTL and the Chr-7 QTL is present in the tomato plant in a heterozygous form and wherein the combination of QTLs confers an increase in the tomato crop plant productivity compared to the productivity of a corresponding tomato crop plant comprising none or one of said QTLs.

47. The tomato crop plant of claim 46, wherein said plant comprises a single exogenous genetic element comprising both the Chr-1 QTL and the Chr-7 QTL.

48. The tomato crop plant of claim 46, wherein said plant comprises an exogenous genetic element comprising the Chr-1 QTL and an additional exogenous genetic element comprising the Chr-7 QTL.

49. The tomato crop plant of claim 46, wherein the Chr-1 QTL comprises a polynucleotide comprising a nucleic acid sequence having at least 96% identity to the nucleic acid sequence of the S. pennellii chromosome 1 from about 100.789953 cM to about 127.0420104 cM or to a part thereof.

50. The tomato crop plant of claim 46, wherein the Chr-1 QTL comprises a polynucleotide comprising a nucleic acid sequence having at least 96% identity to a nucleic acid sequence selected from the group consisting of SEQ ID NO:1, SEQ ID NO:2 and any combination thereof.

51. The tomato crop plant of claim 46, wherein the Chr-1 QTL comprises a marker amplified by a pair of primers comprising SEQ ID NO:3 and SEQ ID NO:4 and wherein said marker is not amenable to digestion by EcoRI.

52. The tomato crop plant of claim 46, wherein the Chr-7 QTL comprises a polynucleotide comprising a nucleic acid sequence having at least 96% identity to the nucleic acid sequence of the S. pennellii chromosome 7 from about 53.3003069 cM to about 56.96038414 cM or to a part thereof.

53. The tomato crop plant of claim 46, wherein the Chr-7 QTL comprises a polynucleotide comprising a nucleic acid sequence having at least 96% identity to a nucleic acid sequence selected from the group consisting of SEQ ID NO:6, SEQ ID NO:7 and any combination thereof.

54. The tomato crop plant of claim 46, wherein the Chr-7 QTL comprises a marker amplified by a pair of primers comprising SEQ ID NO:8 and SEQ ID NO:9 and wherein said marker is not amenable to digestion by HphI.

55. A seed of the tomato crop plant of claim 46, wherein a plant grown from the seed comprises within its genome one or more exogenous genetic elements comprising a QTL derived from chromosome 1 (Chr-1 QTL) and a QTL derived from chromosome 7 (Chr-7 QTL) of Solanum pennellii Lost Accession LA5240, wherein each of the Chr-1 QTL and the Chr-7 QTL is present in said plant in a heterozygous form and wherein the combination of QTLs confers an increase in crop productivity compared to the productivity of a corresponding plant comprising none or one of said QTLs.

56. A cell or a tissue culture obtained from the plant of claim 46, wherein a plant developed from the cell or tissue culture comprises within its genome one or more exogenous genetic elements comprising a QTL derived from chromosome 1 (Chr-1 QTL) and a QTL derived from chromosome 7 (Chr-7 QTL) of Solanum pennellii Lost Accession LA5240, wherein each of the Chr-1 QTL and the Chr-7 QTL is present in said plant in a heterozygous form and wherein the combination of QTLs confers an increase in crop productivity compared to the productivity of a corresponding plant comprising none or one of said QTLs.

57. A DNA construct comprising an isolated polynucleotide comprising a nucleic acid sequence having at least 96% identity to the nucleic acid sequence of S. pennellii LA5240 chromosome 1 between positions 100.789953 cM and 127.0420104 cM or to a part thereof and at least one regulatory element operably linked to the isolated polynucleotides.

58. A DNA construct comprising an isolated polynucleotide comprising a nucleic acid sequence having at least 96% identity to the nucleic acid sequence of S. pennellii LA5240 chromosome 7 between positions 53.3003069 cM and 56.96038414 cM or to a part thereof and at least one regulatory element operably linked to the isolated polynucleotides.

59. A method for producing a tomato crop plant having an enhanced crop productivity, the method comprising introducing into the plant genome one or more genetic elements comprising a QTL derived from chromosome 1 (Chr-1 QTL) and a QTL derived from chromosome 7 (Chr-7 QTL) of Solanum pennellii Lost Accession LA5240, wherein the combination of the QTLs confers to the tomato plant an increase in crop productivity compared to the productivity of a corresponding tomato plant comprising none or one of said QTLs.

60. The method of claim 59 wherein each of the Chr-1 QTL and the Chr-7 QTL is present on a separate genetic element.

61. The method of claim 60, wherein each of the genetic elements is introduced by introgression from a donor tomato plant to a different recipient tomato plant, thereby producing a recipient tomato plant comprising Chr-1 QTL and a recipient tomato plant comprising Chr-7 QTL.

62. The method of claim 61, wherein said method further comprises crossing the recipient tomato plant comprising Chr-1 QTL and the recipient tomato plant comprising Chr-7 QTL to form a progeny comprising the epistatic combination of Chr-1 QTL and Chr-7 QTL.

63. The method of claim 59, wherein said method comprises introducing to the plant A DNA construct comprising: (i) an isolated polynucleotide comprising a nucleic acid sequence having at least 96% identity to the nucleic acid sequence of S. pennellii LA5240 chromosome 1 between positions 100.789953 cM and 127.0420104 cM or to a part thereof and at least one regulatory element operably linked to the isolated polynucleotides; or (ii) a DNA construct comprising an isolated polynucleotide comprising a nucleic acid sequence having at least 96% identity to the nucleic acid sequence of S. pennellii LA5240 chromosome 7 between positions 53.3003069 cM and 56.96038414 cM or to a part thereof and at least one regulatory element operably linked to the isolated polynucleotides.

64. A method for identifying and selecting a tomato plant characterized by an increased crop productivity, comprising the steps of:
a. providing a plurality of tomato crop plants;
b. examining a nucleic acid sample obtained from each of the plurality of the tomato crop plant for the presence of a Chr-1 QTL and Chr-7 QTL, thereby identifying a population comprising both QTLs; and
c. examining the population comprising both QTLs for enhanced crop productivity compared to a corresponding plant comprising none or one of said QTLs or to a pre-determined productivity score value;
thereby selecting a tomato plant characterized by an increased crop productivity compared to the control plant.

65. The method of claim 64, wherein examining the nucleic acid sample for the presence of the Chr-1 QTL comprises identifying at least one nucleic acid sequence selected from the group consisting of SEQ ID NO:1, SEQ ID NO:2 and SEQ ID NO:5 and examining the nucleic acid sample for the presence of a Chr-7 QTL comprises identifying at least one nucleic acid sequence selected from the group consisting of SEQ ID NO:6, SEQ ID NO:7 and SEQ ID NO: 10.


SEQUENCE LISTING (10 sequences)

SEQ ID NO: 1 - DNA, 1136 bp, Solanum pennellii
  source 1..1136: mol_type = genomic DNA
taatttggtgccaaatcaagttgacgcatgccgacgacattccggtaatgtagacaagcaaagctccgtcagcactcagccccacaataatagaaagtcacaatattctcttcaaaattattttataaaccaaaaccctcaactcccgccgcagggtttctctcctccgcactccgcagccgcattcatctttacagaaaagctccaagggatcgaagagagagccatggcgcagtcactagagctattattgattcaatttctgatgcctgacaatgatgctagaagacaagctgaggatcaaataaagcgtcttgcaaaggatcctcaggtggttccatcactcatacatcatctccgtactgccaaaacgcctaatgttaggcagcttgcggcggtacttctccgtaagaagatcactggtcattgggctaagctatctcctcaacaccgtcagcttgtgaaacagtccttaattgagagcatcactgtggaacacaggtcctttgattcattaccctcactttttagtttcaattgtactagtccttttcccgtattcagtgcaataattgcaccatacattttgctatttattgatgattagtattgtgagtatatttgctgcaactaattttctaggcgctctatggagtttggcatgcgcttttgtatccgcatcttttttcaaaatatacttactttataaagtctgctggattagaatgaactatagttcatacaattctcctattctttttgagtaattcagttttctcttgtgtctgagaatgactttctgatcaaactatagaataaggttttgcaggactaagagactttttgttgtgaaagagtatttgactgatgaagtcaattttgttaactgattattcttataataatggatagaagaatcgctgatttttcaagcaagccaattataacagagtttactttctgtggtaattactccgtgtgcgtctgtgtgagaaaaaaaaatttagatcacacaaccttcacaatttttttcccggagatgcattgaattgccttacttggaagctgttactaggcctccaatggaagttttaaaagatgatattttgcctgcaaattatactgatctaatgcga

SEQ ID NO: 2 - DNA, 1479 bp, Solanum pennellii
  source 1..1479: mol_type = genomic DNA
aacacaagtgcattcagatccgggcatgtgtcggttctaggcgtcgaagtcgtaggagcagaacgccattcttcctttgagatatcccaccacgaatagttgccgtttctggattgcaaagttcacatatattgatgaagaggtgaggcaacatgtcagcatctcaagcgctaggcggtccttcccgctgtggctgggtactaggtccgtctcttgataagatcatcaagaacgtcgcctggaggaaacactcgcagctcgtcgccgcctgcaagtccgctctcgacaagcttgattccgtagttgacgaccctgtcgatccagcttcctgcacccctctctatggtctctctacttctgatgccgatttcgtcttgcaacccctcatcatggctcttgattcttcctctcctaaagttgttgaacctgcactcgattgctccttcaggttgttctccttgggcttgatccgctgcgagatcgacacccccactcctacccccagccccagccataacccaagttcccactcccacattttccgcctaatagattccgtctgtaagtgcggggctcttggggacgaagccatcgagctagctgtactcagggtcttgctctctgccattcgatctccctacgttcttgtcagaggtgattgccttgtccatattgttaggtcttgttacaatgtctatctcgggggaatgaatggcaccaatcagatctgtgccaagtccgttcttgctcagatgatgataattgttttcaccagagtggaggagaattccatgatagtggacttcaagaccgtctctgtagctgaactcttggagttcacggatagaaatttgaacgagggaagctccatacagatcgcccagaatttcctcaatgagattgtggatgtcaagtccaaagaaggcattgctgaatcaaagttgtgtttgcagttggagaatgacaacagtgaaaagaagggtgagctcattgatggagagccgggtgaaggagctgatctgagtggttatagtaagattagagaggatgggttcatgctcttcaagaatttgtgcaagttgtctatgaagttttcatcacaagagcacgcagatgacaatattttgttaagggggaaagtactatcattggagctcctaaaggttatcatggataatgctggtcctatttggcgctcaaatgagaggttatttcatttctattactacatttgctgattgcttgtattttttttgttacacagtggtctgcctattggactgctgatcacatggccagggaaactagttttgtgagtcgagaagttaaccattgtcaccagtttgacaataattttttccaattccttaataattttttttgtctacaatgaaaatctagtgaataaatatgatagtttgcctcccattgaagaaaatctctaatggtgtagaatgtatttttgttcttacatgaagg

SEQ ID NO: 3 - DNA, 24 bp, synthetic construct
  source 1..24: mol_type = other DNA
ctatcactgaagcaactagtgagg

SEQ ID NO: 4 - DNA, 25 bp, synthetic construct
  source 1..25: mol_type = other DNA
cgttgttggtgaatatgagcttcac

SEQ ID NO: 5 - DNA, 729 bp, Solanum pennellii
  source 1..729: mol_type = genomic DNA
ctatcactgaagcaactagtgaggtgaacatgaattatgtacctcaacaccattgtaaaccatgtcataagctaaggcacgcgcagaagagaggtcatttatatcttctggattaggtgcggtgaaaggatgatgcaaagcctgtaggaaaattatggtcacttcacataatgaaatatcataacagagaaacagattaatcttagaaaaaacgtcattatttgattgccacagtttttgagaattagtagtgtttatttggttgcaacacttaaaggcaaaatcaaggatatcctcgctagatcaatcctaataattaatgtaaggcacatcccagctcatgttcttcaaaagcagagactcaaatttcttctcatcttcaaagagaaatgtgacaaacaacccaagttactgttacacatctaaaagttctatttgagtttctcaattttttaacaaataagcaatagttttctagctactggtgagagctctaaaggtgaaattcttgattggtatgaatttattgacctgtgctttctaggtcttgatatgtaagagctttcaggtactgaaaacggaacgcatattagacaagtcaaggtcatgattatagaaaatggacaggccattaaagatcaggattgtaccttctccagctagtaaggtcgtaacaggtaagaataataccacataaaataattgtgaagctcatattcaccaacaacg

SEQ ID NO: 6 - DNA, 1560 bp, Solanum pennellii
  source 1..1560: mol_type = genomic DNA
ctgcttcctttcttcatttgggaattagggagaaaggcggttgctgaacatttgagcaaagaatccatacgaatatatatacacttttttttttcttcttcttcctgtagctctcggcgattgtaattgaagaatccatttgcgaagaaaacgaagaaccctatggagcttctccgatcaaaccttgctcgtgttcggattccagagccgactactcgtatctacaagcacgagtgctgcatttctttcgatactccggtaaatcttcaattccaatttggaatgttcattttgctctaattttacttcattattcgtttgtttcgttgttcctagttgttacgattgtagatgttatttggactcgtccagcatagtttgcaaggctgtaattctcgtccttgtgccatttttggtaagagaagttgtatgttaattcggtagggtttaggagcttatactagggtttatagttcaatcagaagttgtatgttaattcggtagggtttaggagcttatactagggtttatagttcaatcgctgctatttagttgtagaactaaaaattttaatttctaaaaacctctattgatattttgaccaaaagcaaaacctttctttgaacttttagtcattctacttatacagggaataagtgatgattcgaacggagtcaaaatagatatggaagatccatatacagtccatgacttgtactaaatattagtcatcctcactcgattttcaagcttgtaattcttagccttgtgcaatttttagtaagttgagtgcatattaagagtttattcttggtttatgaatcatggctcactccgattgaagttgagcctgcagttgattagtactgatgagtgatttgtctatcaagttgtagaacagactcaaaatatagtgtagtagatatagagaattcgtatggccaaccccagctagtttaggattgacatattagtagtagttgtagtggtatagccccaatgaaatgtcatgctgatgctggcttatcggtttgatcttttccatctgtgttcactaatgctcatttaggaaatatatcttcacttatgaagcagttgctattgatttagctacaatttgtaacattttctttgctgtttttccagaaatccgatggcgggctgtttgttgatatgagtacttttcttgcatttggaagggattgtgttgattggaactatgagaagactgggaaccctgtttatttgcatataaagcagacaaagaaggcagatgctgaagatagaccgtccaaaaaacccactctcttggctataggtacttaaatatttcacatatagctaaaagcttccaaaactaggccaaaaaagaaaaaaagaagaataaagagccatataagttcgcattttctgatatttatcatttgacagagttaagcctacccttactaatttgaggttgaagcttagttgatagattgacttggaatatgcctttctccttctaggtttagacggagggtttgataacagtgaaccccagtacgaagaattctatga

SEQ ID NO: 7 - DNA, 1498 bp, Solanum pennellii
  source 1..1498: mol_type = genomic DNA
tactgagattcatgtagccgatcctaattcacctagtattgatgcatagttattgttgttccatataactacaggaatgatgatttctgtctctctctctaatcaagcatcttaatatagggggcttaataagaatcatgttacaaggagaacttcaagctatcatcttatgaagtagtaattcctaattcacttgtttgtttcttgcagatggttgagccagttttgagggccgcgtcgtatccttcttacttcttttttgcacactaatccttcattttttatgttgattatgggacaagatttacttaaataataggaagagattaagttgttcattttctttcatagctctttgggtgtgttaggtgattggaataaatgagggtatgttttaaaatacagtgaatgcttatttagctacatataacaaaaccttttctgttctacaaattactagagttctgcacagctgatttgccgtaaacatattggtttgataaattggtgttgttggatattaattgtacttcctttcttccttgtttttttccatatattcataattgcacatgtcctatcttcgtctcatttggtaagatgactttgtttttcatctgtgctgtttgcttttcatttcaaagtaaacacttgatcaatgttcctgttttatgtaaattccaatgtggatttttagccattaatgattgtttctacccttaacagtagacagggagatatctacacttcttgcacgatcattttttatgggattttcgctgacagtattgtccttgcttgcccgaataagagttctagttcagcaagtaagtattctcagtataattttatcattttcagaactgtgaaatggtatgtgcgctcatgaagaatgacaccattttaaagctcattccttcgtcatacttcttatagtaaaggtcttgcacatgggttgcgttcttttgaaatttgagtttgttgaaggtttaatttccagtataatacactggagtcatgttagtttctggagcttattatatggagtaactagtctaactcaagagtcaaggggactgggtcatgtatgacatgggactttttgctcttctaggagcatgcaatatttaaggagttgtaaatcttatgttgagcaaattttctaaggatatctagtgattgaacaggacatttaatcaaaatttctctattcattgcagatgcttctagatgttgtttgtgtattcaacaatgtttcctctttatcacagagggaacaagccatcaaattgactcaagatggatttgaggttgtttctttgctactgaccactctgtttttctgatgcaatgcaagaaattcttttccttccttatattccctttgtttactagcttttttgattggtttttaattagttctgttaaggtcaagaaatggttggtaaagttatgttgagataggaaaattgattgtactttcttgatagtcgttaat

SEQ ID NO: 8 - DNA, 22 bp, synthetic construct
  source 1..22: mol_type = other DNA
atggatcgatcggctctgatac

SEQ ID NO: 9 - DNA, 24 bp, synthetic construct
  source 1..24: mol_type = other DNA
ggtagtcaaagtttgaccgacctt

SEQ ID NO: 10 - DNA, 586 bp, Solanum pennellii
  source 1..586: mol_type = genomic DNA
atggatcgatcggctctgataccaaaaacagaagagcccaacccaaaagactagcctgattggtgggagagccatttagcctataattccattaacatttctatttttatcagatgagggacaactagcatataacactctaccttcccatcgggaattgaacccaaaaacttttccaacagcaccatcaaatactcaatttccaacccctgtaactggtttctaaagtttggcatctgcgtcaaagaatctttatacaatttgtattattgcccacaaatacctcttgctatttttctttttgttctacgcacaaaaggatcttggttatttgaattgagtggtgggaaggtaaacatttctctggatggaaattcacaagcaacaagcaccaaaacacaagttccctaggttgtaatgcatttgattacatagtcacatattacttagaaacttctttaaggtcggtcaaactttgactacctttatttgacccctcatgcaacccccacactcaaacccaccgaaattacaaggacatggcaacatcattttgtgccatccatcttcagactcccatcctcat